(19) United States
(12) Patent Application Publication
(10) Pub. No.: US 2020/0016267 A1
(43) Pub. Date: Jan. 16, 2020

(54) AQUEOUS ANTI-PD-L1 ANTIBODY FORMULATION

(71) Applicants: Merck Patent GMBH, Darmstadt (DE); Pfizer Inc., New York, NY (US)

(72) Inventors: Gianluca Rinaldi, Monterotondo (IT); Silvia Fratarcangeli, Ceprano FR (IT); Michael James Shopik, Roma (IT); Alessandra Del Rio, Roma (IT)

(21) Appl. No.: 16/491,502

(22) PCT Filed: Mar. 6, 2018
(86) PCT No.: PCT/EP2018/055404

§ 371 Date: Sep. 5, 2019

(30) Foreign Application Priority Data
Mar. 6, 2017 (EP) 17159354.4

(51) Int. Cl. A61K 39/395; C07K 16/28; A61K 9/00

(52) U.S. Cl. CPC A61K 39/39591; C07K 16/2827; A61K 2039/54; C07K 2317/21; C07K 2317/76; A61K 9/0019

(57) ABSTRACT

The present invention relates to a novel anti-PD-L1 antibody formulation. In particular, the invention relates to an aqueous pharmaceutical formulation of the anti-PD-L antibody Avelumab.

The present invention relates to a novel anti-PD-L1 antibody formulation. In particular, the invention relates to an aqueous pharmaceutical formulation of the anti-PD-L1 antibody Avelumab.

### BACKGROUND OF THE INVENTION

The programmed death 1 (PD-1) receptor and PD-1 ligands 1 and 2 (PD-L1, PD-L2) play integral roles in immune regulation. Expressed on activated T cells, PD-1 is activated by PD-L1 and PD-L2 expressed by stromal cells, tumor cells, or both, initiating T-cell death and localized immune suppression (Dong H, Zhu G, Tamada K, Chen L. B7-H1, a third member of the B7 family, co-stimulates T-cell proliferation and interleukin-10 secretion. Nat Med 1999; 5:1365-69; Freeman G J, Long A J, Iwai Y, et al. Engagement of the PD-1 immunoinhibitory receptor by a novel B7 family member leads to negative regulation of lymphocyte activation. J Exp Med

2000; 192:1027-34; Dong H, Strome S E, Salomao D R, et al. Tumor-associated B7-H1 promotes T-cell apoptosis: a potential mechanism of immune evasion. Nat Med 2002; 8:793-800. Erratum, Nat Med 2002; 8:1039; Topalian S L, Drake C G, Pardoll D M. Targeting the PD-1/B7-H1 (PD-L1) pathway to activate anti-tumor immunity. Curr Opin Immunol 2012; 24:207-12), potentially providing an immune-tolerant environment for tumor development and growth. Conversely, inhibition of this interaction can enhance local T-cell responses and mediate antitumor activity in nonclinical animal models (Dong H, Strome S E, Salomao D R, et al. Nat Med 2002; 8:793-800. Erratum, Nat Med 2002; 8:1039; Iwai Y, Ishida M, Tanaka Y, et al. Involvement of PD-L1 on tumor cells in the escape from host immune system and tumor immunotherapy by PD-L1 blockade. Proc Natl Acad Sci USA 2002; 99:12293-97). In the clinical setting, treatment with antibodies that block the PD-1-PD-L1 interaction have been reported to produce objective response rates of 7% to 38% in patients with advanced or metastatic solid tumors, with tolerable safety profiles (Hamid O, Robert C, Daud A, et al. Safety and tumor responses with lambrolizumab (Anti-PD-1) in melanoma. N Engl J Med 2013; 369:134-44; Brahmer J R, Tykodi S S, Chow L Q, et al. Safety and activity of anti-PD-L1 antibody in patients with advanced cancer. N Engl J Med 2012; 366(26):2455-65; Topalian S L, Hodi F S, Brahmer J R, et al. Safety, activity, and immune correlates of anti-PD-1 antibody in cancer. N Engl J Med 2012; 366(26):2443-54; Herbst R S, Soria J-C, Kowanetz M, et al. Predictive correlates of response to the anti-PD-L1 antibody MPDL3280A in cancer patients. Nature 2014; 515:563-67). Notably, responses appeared prolonged, with durations of 1 year or more for the majority of patients.

Avelumab (also known as MSB0010718C) is a fully human monoclonal antibody of the immunoglobulin (Ig) G1 isotype. Avelumab selectively binds to PD-L1 and competitively blocks its interaction with PD-1.

Compared with anti-PD-1 antibodies that target T-cells, Avelumab targets tumor cells, and therefore is expected to have fewer side effects, including a lower risk of autoimmune-related safety issues, as blockade of PD-L1 leaves the PD-L2-PD-1 pathway intact to promote peripheral self-tolerance (Latchman Y, Wood C R, Chernova T, et al. PD-L1 is a second ligand for PD-1 and inhibits T cell activation. Nat Immunol 2001; 2(3):261-68).

Avelumab is currently being tested in the clinic in a number of cancer types including non-small cell lung cancer, urothelial carcinoma, mesothelioma, Merkel cell carcinoma, gastric or gastroesophageal junction cancer, ovarian cancer, and breast cancer.

The amino acid sequences of Avelumab and sequence variants and antigen binding fragments thereof, are disclosed in WO2013079174, where the antibody having the amino acid sequence of Avelumab is referred to as A09-246-2. Also disclosed are methods of manufacturing and certain medical uses.

Further medical uses of Avelumab are described in WO2016137985, WO2016181348, WO2016205277, PCT/US2016/053939, U.S. patent application Ser. No. 62/423,358. WO2013079174 also describes in section 2.4 a human aqueous formulation of an antibody having the amino acid sequence of Avelumab. This formulation comprises the antibody in a concentration of 10 mg/ml, methionine as an antioxidant and has a pH of 5.5. Avelumab formulations not comprising an antioxidant are described in PCT/EP2016/002040.

A formulation study for an aglycosylated anti-PD-L1 antibody of the IgG1 type is described in WO2015048520, where a formulation with a pH of 5.8 was selected for clinical studies.

### DESCRIPTION OF THE INVENTION

As Avelumab is generally delivered to a patient via intravenous infusion, and is thus provided in an aqueous form, the present invention relates to further aqueous formulations that are suitable to stabilize Avelumab with its post-translational modifications, and at higher concentrations as disclosed in WO2013079174.

FIG. 1a (SEQ ID NO:1) shows the full length heavy chain sequence of Avelumab, as expressed by the CHO cells used as the host organism.

It is frequently observed, however, that in the course of antibody production the C-terminal lysine (K) of the heavy chain is cleaved off. Located in the Fc part, this modification has no influence on the antibody-antigen binding. Therefore, in some embodiments the C-terminal lysine (K) of the heavy chain sequence of Avelumab is absent. The heavy chain sequence of Avelumab without the C-terminal lysine is shown in FIG. 1b (SEQ ID NO:2).

FIG. 2 (SEQ ID NO:3) shows the full length light chain sequence of Avelumab.

A post-translational modification of high relevance is glycosylation.

Most of the soluble and membrane-bound proteins that are made in the endoplasmatic reticulum of eukaryotic cells undergo glycosylation, where enzymes called glycosyltransferases attach one or more sugar units to specific glycosylation sites of the proteins. Most frequently, the points of attachment are NH₂ or OH groups, leading to N-linked or O-linked glycosylation.

This applies also to proteins, such as antibodies, which are recombinantly produced in eukaryotic host cells. Recombinant IgG antibodies contain a conserved N-linked glycosylation site at a certain asparagine residue of the Fc region in the CH2 domain. There are many known physical functions of N-linked glycosylation in an antibody such as affecting its solubility and stability, protease resistance, binding to Fc receptors, cellular transport and circulatory half-life in vivo (Hamm M. et al.,

Pharmaceuticals 2013, 6, 393-406). IgG antibody N-glycan structures are predominantly biantennary complex-type structures, comprising b-D-N-acetylglucosamine (GlcNac), mannose (Man) and frequently galactose (Gal) and fucose (Fuc) units.

In Avelumab the single glycosylation site is Asn300, located in the CH2 domain of both heavy chains. Details of the glycosylation are described in Example 1.

Since glycosylation affects the solubility and stability of an antibody, it is prudent to take this parameter into account when a stable, pharmaceutically suitable formulation of the antibody is to be developed.

Surprisingly, it has been found by the inventors of the present patent application that it is possible to stabilize Avelumab, fully characterized by its amino acid sequence and its post-translational modifications, in a number of aqueous formulations without the presence of an antioxidant, at pH values even below 5.2.

### FIGURES

FIG. 1a: Heavy chain sequence of Avelumab (SEQ ID NO:1)

FIG. 1b: Heavy chain sequence of Avelumab, lacking the C-terminal K (SEQ ID NO:2)

FIG. 2: Light chain sequence of Avelumab (SEQ ID NO:3)

FIG. 3: Secondary structure of Avelumab

FIG. 4: 2AB HILIC-UPLC Chromatogram of Avelumab Glycans

FIG. 5: Numbering of the peaks of FIG. 4

### DEFINITIONS

Unless otherwise stated, the following terms used in the specification and claims have the following meanings set out below.

References herein to “Avelumab” include the anti-PD-L1 antibody of the IgG1 type as defined in WO2013079174 by its amino acid sequence, and as defined in the present patent application by its amino acid sequence and by its post-translational modifications. References herein to “Avelumab” may include biosimilars which, for instance, may share at least 75%, suitably at least 80%, suitably at least 85%, suitably at least 90%, suitably at least 95%, suitably at least 96%, suitably at least 97%, suitably at least 98% or most suitably at least 99% amino acid sequence identity with the amino acid sequences disclosed in WO2013079174. Alternatively or additionally, references herein to “Avelumab” may include biosimilars which differ in the post-translational modifications, especially in the glycosylation pattern, herein disclosed.

The term “biosimilar” (also known as follow-on biologics) is well known in the art, and the skilled person would readily appreciate when a drug substance would be considered a biosimilar of Avelumab. The term “biosimilar” is generally used to describe subsequent versions (generally from a different source) of “innovator biopharmaceutical products” (“biologics” whose drug substance is made by a living organism or derived from a living organism or through recombinant DNA or controlled gene expression methodologies) that have been previously officially granted marketing authorisation. Since biologics have a high degree of molecular complexity, and are generally sensitive to changes in manufacturing processes (e.g. if different cell lines are used in their production), and since subsequent follow-on manufacturers generally do not have access to the originator's molecular clone, cell bank, know-how regarding the fermentation and purification process, nor to the active drug substance itself (only the innovator's commercialized drug product), any “biosimilar” is unlikely to be exactly the same as the innovator drug product.

Herein, the term “buffer” or “buffer solution” refers to a generally aqueous solution comprising a mixture of an acid (usually a weak acid, e.g. acetic acid, citric acid, imidazolium form of histidine) and its conjugate base (e.g. an acetate or citrate salt, for example, sodium acetate, sodium citrate, or histidine) or alternatively a mixture of a base (usually a weak base, e.g. histidine) and its conjugate acid (e.g. protonated histidine salt). The pH of a “buffer solution” will change very only slightly upon addition of a small quantity of strong acid or base due to the “buffering effect” imparted by the “buffering agent”.

Herein, a “buffer system” comprises one or more buffering agent(s) and/or an acid/base conjugate(s) thereof, and more suitably comprises one or more buffering agent(s) and an acid/base conjugate(s) thereof, and most suitably comprises one buffering agent only and an acid/base conjugate thereof. Unless stated otherwise, any concentrations stipulated herein in relation to a “buffer system” (i.e. a buffer concentration) suitably refers to the combined concentration of the buffering agent(s) and/or acid/base conjugate(s) thereof. In other words, concentrations stipulated herein in relation to a “buffer system” suitably refer to the combined concentration of all the relevant buffering species (i.e. the species in dynamic equilibrium with one another, e.g. citrate/citric acid). As such, a given concentration of a histidine buffer system generally relates to the combined concentration of histidine and the imidazolium form of histidine. However, in the case of histidine, such concentrations are usually straightforward to calculate by reference to the input quantities of histidine or a salt thereof. The overall pH of the composition comprising the relevant buffer system is generally a reflection of the equilibrium concentration of each of the relevant buffering species (i.e. the balance of buffering agent(s) to acid/base conjugate(s) thereof).

Herein, the term “buffering agent” refers to an acid or base component (usually a weak acid or weak base) of a buffer or buffer solution. A buffering agent helps maintain the pH of a given solution at or near to a pre-determined value, and the buffering agents are generally chosen to complement the pre-determined value. A buffering agent is suitably a single compound which gives rise to a desired buffering effect, especially when said buffering agent is mixed with (and suitably capable of proton exchange with) an appropriate amount (depending on the pre-determined pH desired) of its corresponding “acid/base conjugate”, or if the required amount of its corresponding “acid/base conjugate” is formed in situ—this may be achieved by adding strong acid or base until the required pH is reached. For example in the sodium acetate buffer system, it is possible to start out with a solution of sodium acetate (basic) which is then acidified with, e.g., hydrochloric acid, or to a solution of acetic acid (acidic), sodium hydroxide or sodium acetate is added until the desired pH is reached.

Generally, a “stabiliser” refers to a component which facilitates maintenance of the structural integrity of the biopharmaceutical drug, particularly during freezing and/or lyophilization and/or storage (especially when exposed to stress). This stabilising effect may arise for a variety of reasons, though typically such stabilisers may act as osmolytes which mitigate against protein denaturation. As used herein, stabilisers can be sugar alcohols (e.g. inositol, sorbitol), disaccharides (e.g. sucrose, maltose), monosaccharides (e.g. dextrose (D-glucose)), or forms of the amino acid lysine (e.g. lysine monohydrochloride, acetate or monohydrate), or salts (e.g. sodium chloride).

Agents used as buffering agents, antioxidants or surfactants according to the invention, are excluded from the meaning of the term “stabilisers” as used herein, even if they may exhibit, i.a. stabilising activity.

Herein, the term “surfactant” refers to a surface-active agent, preferably a nonionic surfactant. Examples of surfactants used herein include polysorbate, for example, polysorbate 80 (polyoxyethylene (80) sorbitan monooleate, also known under the tradename Tween 80); polyoxyl castor oil, such as polyoxyl 35 castor oil, made by reacting castor oil with ethylene oxide in a molar ratio of 1:35, also known under the tradename Kolliphor ELP; or Kollidon 12PF or 17PF, which are low molecular weight povidones (polyvinylpyrrolidones), known under the CAS number 9003-39-8 and having slightly different molecular weights (12PF: 2000-3000 g/mol, 17PF: 7000-11000 g/mol).

Agents used as buffering agents, antioxidants or stabilisers according to the invention, are excluded from the meaning of the term “surfactants” as used herein, even if they may exhibit, i.a. surfactant activity.

Herein, the term “stable” generally refers to the physical stability and/or chemical stability and/or biological stability of a component, typically an active or composition thereof, during preservation/storage.

Herein, the term “antioxidant” refers to an agent capable of preventing or decreasing oxidation of the biopharmaceutical drug to be stabilized in the formulation. Antioxidants include radical scavengers (e.g. ascorbic acid, BHT, sodium sulfite, p-amino benzoic acid, glutathione or propyl gallate), chelating agents (e.g. EDTA or citric acid) or chain terminators (e.g. methionine or N-acetyl cysteine).

Agents used as buffering agents, stabilisers or surfactants according to the invention, are excluded from the meaning of the term “antioxidants” as used herein, even if they may exhibit, i.a. antioxidative activity.

A “diluent” is an agent that constitutes the balance of ingredients in any liquid pharmaceutical composition, for instance so that the weight percentages total 100%. Herein, the liquid pharmaceutical composition is an aqueous pharmaceutical composition, so that a “diluent” as used herein is water, preferably water for injection (WFI).

Herein, the term “particle size” or “pore size” refers respectively to the length of the longest dimension of a given particle or pore. Both sizes may be measured using a laser particle size analyser and/or electron microscopes (e.g. tunneling electron microscope, TEM, or scanning electron microscope, SEM). The particle count (for any given size) can be obtained using the protocols and equipment outlined in the Examples, which relates to the particle count of sub-visible particles.

Herein, the term “about” refers to the usual error range for the respective value readily known to the skilled person in this technical field. Reference to “about” a value or parameter herein includes (and describes) embodiments that are directed to that value or parameter per se. In case of doubt, or should there be no art recognized common understanding regarding the error range for a certain value or parameter, “about” means±5% of this value or parameter.

Herein, the term “percent share” in connection with glycan species refers directly to the number of different species. For example the term “said FA2G1 has a share of 25%-41% of all glycan species” means that in 50 antibody molecules analysed, having 100 heavy chains, 25-41 of the heavy chains will exhibit the FA2G1 glycosylation pattern.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

Aqueous Anti-PD-L1 Antibody Formulation

In a first aspect, the invention provides a novel aqueous pharmaceutical antibody formulation, comprising:

(i) Avelumab in a concentration of 1 mg/mL to 30 mg/mL as the antibody;
(ii) glycine, succinate, citrate phosphate or histidine in a concentration of 5 mM to 35 mM as the buffering agent;
(iii) lysine monohydrochloride, lysine monohydrate, lysine acetate, dextrose, sucrose, sorbitol or inositol in a concentration of 100 mM to 320 mM as the stabiliser;
(iv) povidone, polyoxyl castor oil or polysorbate in a concentration of 0.25 mg/mL to 0.75 mg/mL, as the surfactant;
wherein the formulation does not comprise methionine, and
further wherein the formulation has a pH of 3.8 to 5.2.

In a preferred embodiment the formulation does not comprise any antioxidant.

In an embodiment the concentration of Avelumab in the said formulation is about 10 mg/mL to about 20 mg/mL.

In yet another embodiment the concentration of glycine, succinate, citrate phosphate or histidine in the said formulation is about 10 mM to about 20 mM.

In further embodiments, in the said formulation, the concentration of lysine monochloride is about 140 mM to about 280 mM, or the concentration of said lysine monohydrate is about 280 mM, or the concentration of the said lysine acetate is about 140 mM.

In yet another embodiment the concentration of dextrose, sucrose, sorbitol or inositol in the said formulation is about 280 mM.

In yet another embodiment the concentration of povidone, polyoxyl castor oil or polysorbate inositol in the said formulation is about 0.5 mg/mL.

In a preferred embodiment the said povidone in the said formulation is the low molecular weight polyvinylpyrrolidone Kollidon 12PF or 17PF of CAS number 9003-39-8.

In another preferred embodiment the said polyoxyl castor oil is Polyoxyl 35 Castor Oil.

In yet another preferred embodiment the said polysorbate is Polysorbate 80.

In a more preferred embodiment, the novel aqueous pharmaceutical antibody formulation, comprises:

(i) Avelumab in a concentration of 1 mg/mL to about 20 mg/mL as the antibody;
(ii) glycine in a concentration of 5 mM to 15 mM as the buffering agent, and not comprising any other buffering agent;
(iii) lysine monohydrochloride, dextrose, sucrose or sorbitol in a concentration of 100 mM to 320 mM as the stabiliser, and not comprising any other stabiliser;
(iv) Kollidon 12PF, polyoxyl 35 castor oil or Polysorbate 80 in a concentration of 0.25 mg/mL to 0.75 mg/mL, as the surfactant, and not comprising any other surfactant;
wherein the formulation has a pH of 3.8 to 4.6, and does not comprise an antioxidant.

In an equally preferred embodiment, the novel aqueous pharmaceutical antibody formulation, comprises:

(i) Avelumab in a concentration of 1 mg/mL to about 20 mg/mL as the antibody;
(ii) succinate in a concentration of 5 mM to 15 mM as the buffering agent, and not comprising any other buffering agent;
(iii) lysine monohydrochloride, dextrose, sucrose or sorbitol in a concentration of 100 mM to 320 mM as the stabiliser, and not comprising any other stabiliser;
(iv) Kollidon 12PF or polyoxyl 35 castor oil in a concentration of 0.25 mg/mL to 0.75 mg/mL, as the surfactant, and not comprising any other surfactant;
wherein the formulation has a pH of 4.9 to 5.2, and does not comprise an antioxidant.

In an equally preferred embodiment, the novel aqueous pharmaceutical antibody formulation, comprises:

(i) Avelumab in a concentration of 1 mg/mL to about 20 mg/mL as the antibody;
(ii) citrate phosphate in a concentration of 10 mM to 20 mM as the buffering agent, and not comprising any other buffering agent;
(iii) lysine monohydrochloride, dextrose, sucrose or sorbitol in a concentration of 100 mM to 320 mM as the stabiliser, and not comprising any other stabiliser;
(iv) Kollidon 12PF or polyoxyl 35 castor oil in a concentration of 0.25 mg/mL to 0.75 mg/mL, as the surfactant, and not comprising any other surfactant;
wherein the formulation has a pH of 3.8 to 4.7, and does not comprise an antioxidant.

In an equally preferred embodiment, the novel aqueous pharmaceutical antibody formulation, comprises:

(i) Avelumab in a concentration of 1 mg/mL to about 20 mg/mL as the antibody;
(ii) glycine in a concentration of about 10 mM as the buffering agent, and not comprising any other buffering agent;
(iii) lysine monohydrochloride in a concentration of about 140 mM as the stabiliser, and not comprising any other stabiliser;
(iv) polyoxyl 35 castor oil in a concentration of about 0.5 mg/mL as the surfactant, and not comprising any other surfactant;
wherein the formulation has a pH of 4.2 to 4.6, and does not comprise an antioxidant.

In a more preferred embodiment, the novel aqueous pharmaceutical antibody formulation, comprises:

(i) Avelumab in a concentration of 1 mg/mL to about 20 mg/mL as the antibody;
(ii) glycine in a concentration of about 10 mM as the buffering agent, and not comprising any other buffering agent;
(iii) lysine acetate in a concentration of about 140 mM as the stabiliser, and not comprising any other stabiliser;
(iv) polyoxyl 35 castor oil in a concentration of about 0.5 mg/mL as the surfactant, and not comprising any other surfactant;
wherein the formulation has a pH of 4.2 to 4.6, and does not comprise an antioxidant.

In an equally preferred embodiment, the novel aqueous pharmaceutical antibody formulation, comprises:

(i) Avelumab in a concentration of 1 mg/mL to about 20 mg/mL as the antibody;
(ii) histidine in a concentration of about 10 mM as the buffering agent, and not comprising any other buffering agent;
(iii) sucrose in a concentration of about 280 mM as the stabiliser, and not comprising any other stabiliser;
(iv) Kollidon 12PF in a concentration of about 0.5 mg/mL as the surfactant, and not comprising any other surfactant;
wherein the formulation has a pH of 4.8 to 5.2, and does not comprise an antioxidant.

In an equally preferred embodiment, the novel aqueous pharmaceutical antibody formulation, comprises:

(i) Avelumab in a concentration of 1 mg/mL to about 20 mg/mL as the antibody;
(ii) succinate in a concentration of about 10 mM as the buffering agent, and not comprising any other buffering agent;
(iii) lysine monohydrochloride in a concentration of about 140 mM as the stabiliser, and not comprising any other stabiliser;
(iv) polyoxyl 35 castor oil in a concentration of about 0.5 mg/mL as the surfactant, and not comprising any other surfactant;
wherein the formulation has a pH of 4.8 to 5.2, and does not comprise an antioxidant.

In a more preferred embodiment of the above described embodiments, the concentration of Avelumab is about 20 mg/ml.

In an even more preferred embodiments the said formulation consists of:

(i) Avelumab in a concentration of 20 mg/mL;
(ii) glycine in a concentration of 10 mM;
(iii) lysine monohydrochloride in a concentration of 140 mM;
(iv) polyoxyl 35 castor oil in a concentration of 0.5 mg/mL;
(v) HCl of NaOH to adjust the pH;
(vi) water (for injection) as the solvent;
and has a pH of 4.4 (±0.1);
or
(i) Avelumab in a concentration of 20 mg/mL;
(ii) glycine in a concentration of 10 mM;
(iii) lysine acetate in a concentration of 140 mM;
(iv) polyoxyl 35 castor oil in a concentration of 0.5 mg/mL;
(v) HCl of NaOH to adjust the pH;
(vi) water (for injection) as the solvent;
and has a pH of 4.4 (±0.1);
or
(i) Avelumab in a concentration of 20 mg/mL;
(ii) histidine in a concentration of 10 mM;
(iii) sucrose in a concentration of 280 mM;
(iv) Kollidon 12PF in a concentration of 0.5 mg/mL;
(v) HCl of NaOH to adjust the pH;
(vi) water (for injection) as the solvent;
and has a pH of 5.0 (±0.1);
or
(i) Avelumab in a concentration of 20 mg/mL;
(ii) succinate in a concentration of 10 mM;
(iii) lysine monohydrochloride in a concentration of 140 mM;
(iv) polyoxyl 35 castor oil in a concentration of 0.5 mg/mL;
(v) HCl of NaOH to adjust the pH;
(vi) water (for injection) as the solvent;
and has a pH of 5.0 (±0.1).

In another preferred embodiment, the formulation has a osmolality between 270 and 330 mOsm/kg.

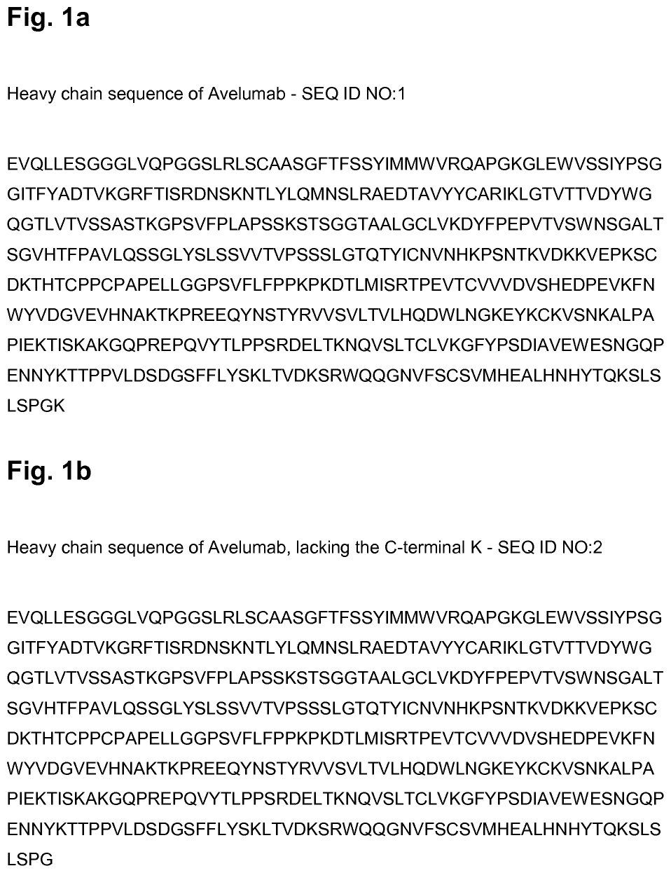

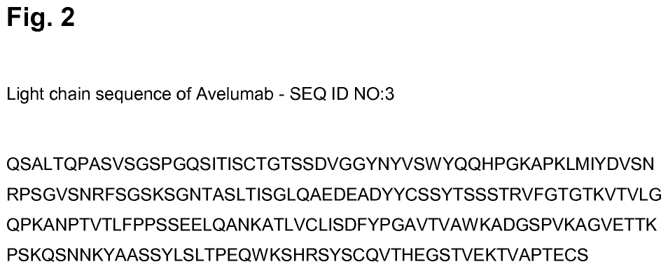

In an embodiment said Avelumab in the formulations as described above has the heavy chain sequence of either FIG. 1a (SEQ ID NO:1) or FIG. 1b (SEQ ID NO:2), the light chain sequence of FIG. 2 (SEQ ID NO:3), and carries a glycosylation on Asn300 comprising FA2 and FA2G1 as the main glycan species, having a joint share of >70% of all glycan species.

In a preferred embodiment, in the Avelumab glycosylation the said FA2 has a share of 44%-54% and said FA2G1 has a share of 25%-41% of all glycan species.

In a preferred embodiment, in the Avelumab glycosylation the said FA2 has a share of 47%-52% and said FA2G1 has a share of 29%-37% of all glycan species.

In a preferred embodiment, in the Avelumab glycosylation the said FA2 has a share of about 49% and said FA2G1 has a share of about 30%-about 35% of all glycan species.

In a preferred embodiment the Avelumab glycosylation further comprises as minor glycan species A2 with a share of <5%, A2G1 with a share of <5%, A2G2 with a share of <5% and FA2G2 with a share of <7% of all glycan species.

In a preferred embodiment, in the Avelumab glycosylation said A2 has a share of 3%-5%, said A2G1 has a share of <4%, said A2G2 has a share of <3% and said FA2G2 has a share of 5%-6% of all glycan species.

In a preferred embodiment, in the Avelumab glycosylation said A2 has a share of about 3.5%-about 4.5%, said A2G1 has a share of about 0.5%-about 3.5%, said A2G2 has a share of <2.5% and said FA2G2 has a share of about 5.5% of all glycan species.

In an embodiment the said Avelumab in the formulation as described above has the heavy chain sequence of FIG. 1b (SEQ ID NO:2).

In an embodiment the Avelumab formulation as described above is for intravenous (IV) administration.

Drug-Delivery Device

In a second aspect the present invention provides a drug delivery device comprising a liquid pharmaceutical composition as defined herein. Suitably the drug delivery device comprises a chamber within which the pharmaceutical composition resides. Suitably the drug delivery device is sterile.

The drug delivery device may a vial, ampoule, syringe, injection pen (e.g. essentially incorporating a syringe), or i.v. (intravenous) bag.

The aqueous pharmaceutical formulations are parenterally administered, preferably via sub-cutaneous injection, intramuscular injection, i.v. injection or i.v. infusion. The most preferred way of administration is i.v. infusion.

In a preferred embodiment, the drug delivery device is a vial containing the formulation as described above.

In a more preferred embodiment the said vial contains 200 mg avelumab in 10 mL of solution for a concentration of 20 mg/mL.

In an even more preferred embodiment the vial is a glass vial.

Medical Treatment

In a third aspect, the invention provides a method of treating cancer comprising administering the formulation as described above to a patient.

In an embodiment the cancer to be treated is selected from non-small cell lung cancer, urothelial carcinoma, bladder cancer, mesothelioma, Merkel cell carcinoma, gastric or gastroesophageal junction cancer, ovarian cancer, breast cancer, thymoma, adenocarcinoma of the stomach, adrenocortical carcinoma, head and neck squamous cell carcinoma, renal cell carcinoma, melanoma, and/or classical Hodgkin's lymphoma.

Methods of Manufacturing

The present invention also provides a method of manufacturing an aqueous pharmaceutical formulation as defined herein. The method suitably comprises mixing together, in any particular order deemed appropriate, any relevant components required to form the aqueous pharmaceutical formulation. The skilled person may refer to the examples or techniques well known in the art for forming aqueous pharmaceutical formulations (especially those for injection via syringe, or i.v. infusion).

The method may involve first preparing a pre-mixture (or pre-solution) of some or all components (optionally with some or all of the diluent) excluding Avelumab, and Avelumab may then itself (optionally with or pre-dissolved in some of the diluent) be mixed with the pre-mixture (or pre-solution) to afford the aqueous pharmaceutical formulation, or a composition to which final components are then added to furnish the final aqueous pharmaceutical formulation. Preferably, the method involves forming a buffer system, suitably a buffer system comprising a buffering agent as defined herein. The buffer system is suitably formed in a pre-mixture prior to the addition of Avelumab. The buffer system may be formed through simply mixing the buffering agent (supplied ready-made) with its acid/base conjugate (suitably in appropriate relative quantities to provide the desired pH—this can be determined by the skilled person either theoretically or experimentally). In the case of an acetate buffer system, this means e.g. mixing sodium acetate with HCl, or mixing acetic acid with NaOH or acetate. The pH of either the pre-mixture of final aqueous pharmaceutical formulation may be judiciously adjusted by adding the required quantity of base or acid, or a quantity of buffering agent or acid/base conjugate.

In certain embodiments, the buffering agent and/or buffer system is pre-formed as a separate mixture, and the buffer system is transferred to a precursor of the aqueous pharmaceutical formulation (comprising some or all components save for the buffering agent and/or buffer system, suitably comprising Avelumab and potentially only Avelumab) via buffer exchange (e.g. using diafiltration until the relevant concentrations or osmolality is reached). Additional excipients may be added thereafter if necessary in order to produce the final liquid pharmaceutical composition. The pH may be adjusted once or before all the components are present.

Any, some, or all components may be pre-dissolved or pre-mixed with a diluent prior to mixing with other components.

The final aqueous pharmaceutical formulation may be filtered, suitably to remove particulate matter. Suitably filtration is through filters sized at or below 1 μm, suitably at 0.22 μm. Suitably, filtration is through either PES filters or PVDF filters, suitably with 0.22 μm PES filters.

The person of skill in the art is well aware how an aqueous pharmaceutical formulation can be used to prepare an IV solution, so that the antibody drug substance can be administered intravenously.

The preparation of the IV solution typically consists of a certain amount of solution being withdrawn from saline bags (e.g. 0.9% or 0.45% saline) with a plastic syringe (PP) and a needle and replaced with aqueous pharmaceutical formulation. The amount of solution replaced will depend on the body weight of the patients.

### ABBREVIATIONS

ANOVA Analysis of variance
CD Circular dichroism
CE-SDS Capillary electrophoresis sodium dodecyl sulfate
cIEF Capillary isoelectrofocusing

### DoE Design of Experiment

### DP Drug Product

### DS Drug Substance

### FT Freeze-thawing

### HMW Higher Molecular Weight

### LMW Low Molecular Weight

### SE-HPLC Size Exclusion High Performance Liquid Chromatography

### OD Optical Density

### PES Polyethersulphone

PVDF Polyvinylidene fluoride

### RH Relative Humidity

SE-HPLC Size-exclusion high performance chromatography

### UV Ultraviolet

### WFI Water for Injection

### EXAMPLES

### Example 1—Structure of Avelumab

1.1 Primary Structure

Avelumab is an IgG with two heavy and two light chain molecules. The amino acid sequences of the two chains are shown in FIGS. 1a (SEQ ID NO:1)/1b (SEQ ID NO:2) and 2 (SEQ ID NO:3), respectively.

1.2 Secondary Structure

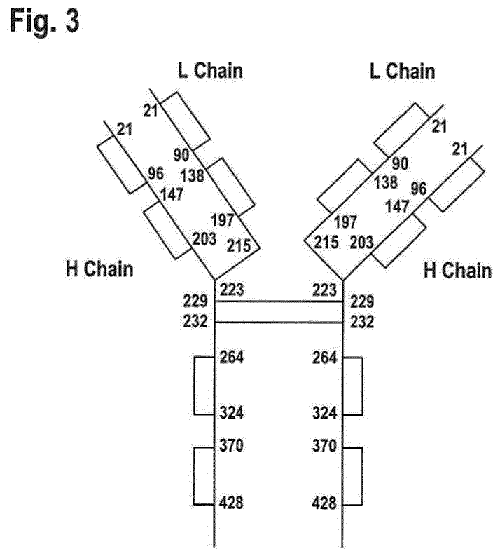

LC-MS and MS/MS methods were used to confirm the intact chains of the molecule and the presence of post-translational modifications to the proteins. The secondary structure of the Avelumab molecule subunits are shown in FIG. 3.

As confirmed by UPLC-Q-TOF mass spectrometry of peptides obtained by trypsin digestion, the disulfide bonds Cys21-Cys96, Cys21-Cys90, Cys147-Cys203, Cys138-Cys197, Cys215-Cys223, Cys229-Cys229, Cys232-Cys232, Cys264-Cys324 and Cys370-Cys428 are forming the nine typical IgG bonding pattern.

1.3 Glycosylation

The molecule contains one N-glycosylation site on Asn300 of the heavy chain. As determined by peptide mapping, the main structure identified by MALDI-TOF was a complex, biantennary type core fucosylated oligosaccaride with zero (G0F), one (G1F), or two galactose (G2F) residues. The main species are G0F and G1F.

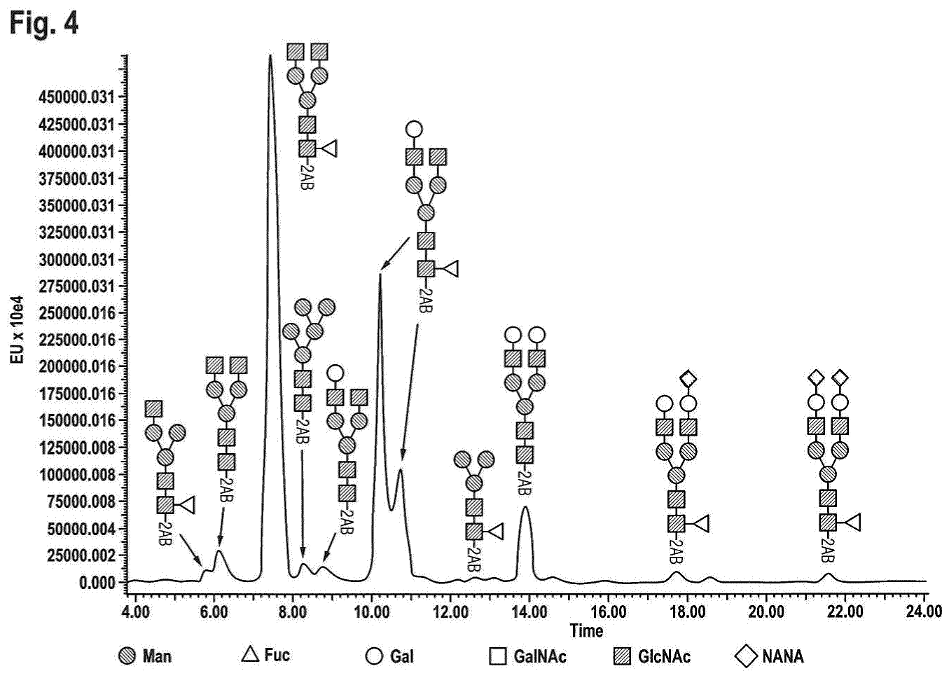

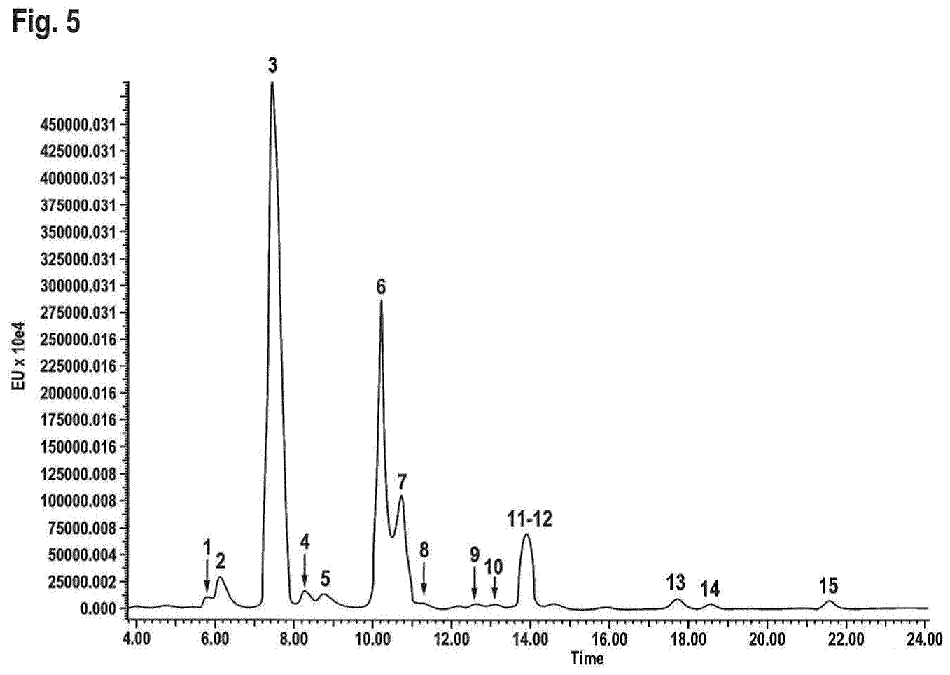

Avelumab glycans fluorescence labeled by 2-aminobenzamide have been analysed by HILIC-UPLC-ESI-Q-TOF. FIG. 4 shows the UPLC profile of the glycan species found.

TABLE 1
Peak identification of 2AB HILIC-UPLC chromatogram

| | RT | Measured | Exppected | | Oxford | Identification |
|---|---|---|---|---|---|---|
| Peak | (min) | MW | MW | Identification | nomenclature | by |
| 1a | 5.99 | 1380.52 (M + H) | 1380.54 (M + H) | 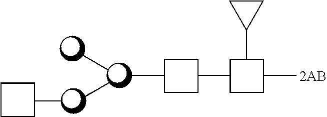 | FA1 | Manually identified by MS |
| 2 | 6.01 | 1437.54 | 1437.56 | 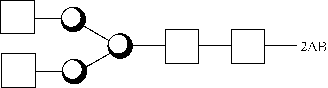 | A2 | Manually identified by MS |
| 3 | 7.02 | 1583.74 (M + H) | 1583.62 (M + H) | 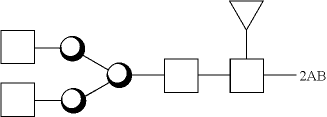 | FA 2 | MS in source fragmentation by GlycoworkBench |
| 4 | 7.77 | 1355.57 (M + H) | 1355.51 (M + H) | 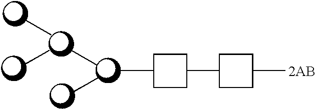 | M5 | Manually identified by MS |
| 5 | 8.16 | 1599.77 (M + H) | 1599.62 (M + H) | 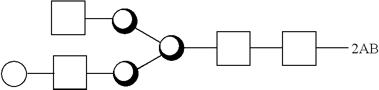 | A2G1 | Manually identified by MS |
| 6 | 9.82 | 1744.79 | 1744.67 | 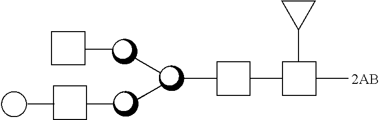 | FA2G1 | MS in source fragmentation by GlycoworkBench |
| | | 1462.90 | 1462.54 | 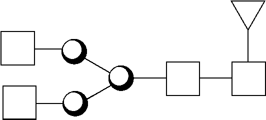 | FA2 freeEnd | GlycoworkBench identified by Ms |
| 7 | 10.07 | 1744.80 | 1744.67 | 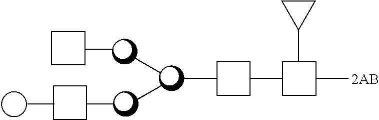 | FA2G1 | MS in source fragmentation by GlycoworkBench |
| | | 1462.91 | 1462.54 | 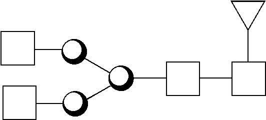 | FA2 freeEnd | GlycoworkBench identified by Ms |
| 8 | 10.44 | 1462.90 | 1462.54 | 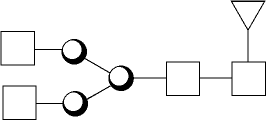 | FA2 freeEnd | GlycoworkBench identified by Ms |
| | | 1744.79 | 1744.67 | 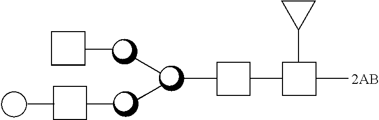 | FA2G1 | Manually identified by MS |
| 9 | 12.15 | 1177.50 (M + H) | 1177.46 (M + H) | 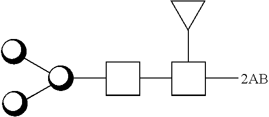 | FM3 | GlycoworkBench identified by Ms |
| 10 | 16.66 | No | No | | | |
| | | ionization | ionization | | | |
| 11 | 13.42 | 1906.33 | 1906.72 | 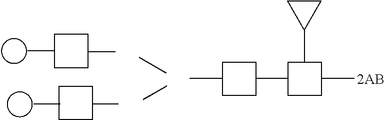 | FA2G2 | MS in source fragmentation by GlycoworkBench |
| | | 1624.71 | 1624.59 | 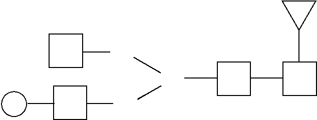 | FA2G1 freeEnd | GlycoworkBench identified by Ms |
| 12 | 13.71 | 954.40 (M + 2H)/2 | 954.36 (M + 2H)/2 | 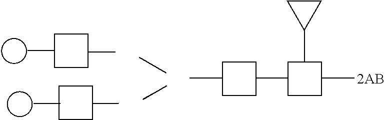 | FA2G2 | Manually identified by MS |
| | | 1626.69 | 1626.61 | 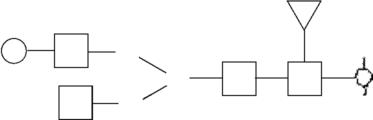 | FA2G1 redEnd | GlycoworkBench identified by Ms |
| 13 | 17.46 | 1099.97 (M + 2H)/2 | 1099.91 (M + 2H)/2 | 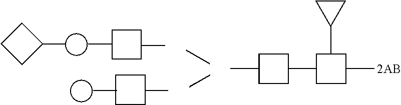 | FA2G2S | MS in source fragmentation by GlycoworkBench |
| 14 | 18.54 | 1079.91 (M + 2H)/2 | 1079.86 (M + 2H)/2 | 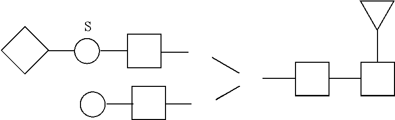 | FA2G2S freeEnd + S (probable- small traces) | Manually identified by MS |
| 15 | 21.04 | 2489.05 | 2488.91 | 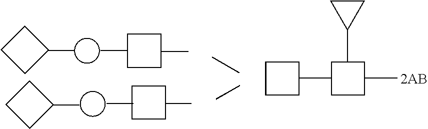 | FA2G2S2 | Manually identified by MS |

The geometric shapes representing the glycan building blocks correspond to the following molecular entities:

| | | | | | |
|---|---|---|---|---|---|
| [symbol] Man | Δ Fuc | ◯ Gal | □ GalNAc | [symbol] GlcNAc | ⋄ NANA |

Man: mannose,
Fuc: fucose,
Gal: galactose,
GalNAc:
N-Acetylgalactosamine,
NANA: sialic acid

The glycan nomenclature used follows the Oxford Notation as proposed by Harvey et al. (Proteomics 2009, 9, 3796-3801). In species containing fucose (FA2, FA2G1, FA2G2), the Fuc-GlcNAc connectivity is α1-6. In species having a terminal GlcNAc, the GlcNAc-Man connectivity is β1-2. In species containing galactose, the Gal-GlcNAc connectivity is β1-4.

The reported chromatographic profile has been integrated and yielded the Glycan Species Distribution of Avelumab as shown in Table 2a.

TABLE 2a

| A2 | FA2 | A2G1 | FA2G1 | A2G2 | FA2G2 | M5** |
|---|---|---|---|---|---|---|
| 3.6 | 48.7 | 3.4 | 35.6 | 2.3 | 5.4 | 1.0 |

**Probably Mannose 5, coelution with biantennary mono-galactosylated species

The glycan mapping analysis confirmed the identification carried out by peptide mapping (that allowed to identify the two main glycan species), in addition secondary and minor species were also characterized by this method, specific for glycan analysis.

In another measurement the following Glycan Species Distribution was observed.

TABLE 2b

| A2 | FA2 | A2G1 | FA2G1 | A2G2 | FA2G2 |
|---|---|---|---|---|---|
| 4.0 | 50.2 | 1.0 | 30.0 | 0.1 | 5.6 |

### Example 2—DoE Screening

A Design of Experiment screening at 20 mg/mL Avelumab assessed the impact of several factors such as varying buffer type/pH, stabilisers, surfactant type and relevant concentration. The study, testing 80 different formulations, led to the selection of the suitable conditions that can maximize protein stability.

Four different buffers were examined in this DoE covering different buffer types and effective pH buffering range:

Amino acid buffers such as Glycine (effective pH 4.0 to 7.5) and Histidine (effective pH 5.0 to 6.6).

Chelating ionic buffer such as Citrate (effective pH 4.0 to 7.5).

Succinate (effective pH 5.0 to 6.0).

Seven stabilisers were selected in the DoE on the basis of their chemical structure.

Included in the DoE were sugars, polyols, salts, and amino acids. The breakdown is as follows:

Sugars: The disaccharides Sucrose and Maltose were selected as well as the monosaccharide Dextrose (D-Glucose).

Sugar alcohols: Two sugar alcohols/polyols were selected for the DoE-Sorbitol and Inositol.

Salt: Sodium chloride was investigated as a stand-alone stabiliser in this DoE.

Amino acid: Lysine, a positively charged amino acid was investigated.

Table 3 lists the samples and their respective compositions.

TABLE 3
DoE screening formulations

| | | | Buffer | | |
|---|---|---|---|---|---|
| Sample | | | Strength | Stabiliser | Surfactant |
| ID | pH | Buffer | (mM) | (280 mM) | (0.5 mg/mL) |
| 1 | 4 | Citrate-phosphate | 10 | Sorbitol | Kollidon 12PF |
| 2 | 4 | Citrate-phosphate | 50 | Dextrose | Tween 40 |
| 3 | 4.5 | Citrate-phosphate | 20 | Dextrose | Tween 40 |
| 4 | 4.5 | Citrate-phosphate | 30 | Inositol | Kollidon 12PF |
| 5 | 4.8 | Citrate-phosphate | 40 | Maltose | Kolliphor ELP |
| 6 | 4.8 | Citrate-phosphate | 40 | Lysine | Tween 80 |
| 7 | 5.2 | Citrate-phosphate | 50 | Dextrose | Kolliphor ELP |
| 8 | 5.2 | Citrate-phosphate | 10 | Sodium chloride | Tween 80 |
| 9 | 5.2 | Citrate-phosphate | 20 | Lysine | Kolliphor ELP |
| 10 | 5.5 | Citrate-phosphate | 20 | Sucrose | Kollidon 12PF |
| 11 | 5.5 | Citrate-phosphate | 30 | Lysine | Tween 40 |
| 12 | 6 | Citrate-phosphate | 20 | Maltose | Tween 40 |
| 13 | 6 | Citrate-phosphate | 30 | Sodium chloride | Kolliphor ELP |
| 14 | 6.5 | Citrate-phosphate | 30 | Dextrose | Tween 80 |
| 15 | 6.5 | Citrate-phosphate | 30 | Sorbitol | Tween 80 |
| 16 | 7 | Citrate-phosphate | 50 | Sucrose | Tween 80 |
| 17 | 7 | Citrate-phosphate | 10 | Lysine | Kollidon 12PF |
| 18 | 7 | Citrate-phosphate | 10 | Inositol | Tween 80 |
| 19 | 7 | Citrate-phosphate | 30 | Sodium chloride | Tween 40 |
| 20 | 7.5 | Citrate-phosphate | 50 | Inositol | Kolliphor ELP |
| 21 | 7.5 | Citrate-phosphate | 50 | Sorbitol | Tween 40 |
| 22 | 4 | Glycine | 10 | Sodium chloride | Kollidon 12PF |
| 23 | 4 | Glycine | 10 | Dextrose | Tween 40 |
| 24 | 4 | Glycine | 30 | Sorbitol | Tween 40 |
| 25 | 4.3 | Glycine | 50 | Sorbitol | Kolliphor ELP |
| 26 | 4.3 | Glycine | 50 | Inositol | Tween 40 |
| 27 | 4.3 | Glycine | 50 | Dextrose | Tween 80 |
| 28 | 4.5 | Glycine | 30 | Sodium chloride | Tween 40 |
| 29 | 4.8 | Glycine | 40 | Lysine | Tween 80 |
| 30 | 4.8 | Glycine | 40 | Maltose | Tween 80 |
| 31 | 5.8 | Glycine | 50 | Lysine | Kollidon 12PF |
| 32 | 5.8 | Glycine | 30 | Maltose | Kollidon 12PF |
| 33 | 6 | Glycine | 30 | Sucrose | Kolliphor ELP |
| 34 | 6.5 | Glycine | 30 | Sodium chloride | Tween 80 |
| 35 | 6.8 | Glycine | 40 | Dextrose | Kolliphor ELP |
| 36 | 6.8 | Glycine | 10 | Inositol | Tween 80 |
| 37 | 6.8 | Glycine | 10 | Sorbitol | Kollidon 12PF |
| 38 | 7 | Glycine | 10 | Lysine | Kolliphor ELP |
| 39 | 7 | Glycine | 10 | Inositol | Tween 40 |
| 40 | 7 | Glycine | 30 | Sodium chloride | Tween 80 |
| 41 | 7.5 | Glycine | 30 | Inositol | Kolliphor ELP |
| 42 | 7.5 | Glycine | 50 | Dextrose | Kollidon 12PF |
| 43 | 7.5 | Glycine | 10 | Sucrose | Kollidon 12PF |
| 44 | 5 | Histidine | 10 | Maltose | Kolliphor ELP |
| 45 | 5 | Histidine | 10 | Sorbitol | Kolliphor ELP |
| 46 | 5 | Histidine | 20 | Dextrose | Kollidon 12PF |
| 47 | 5.2 | Histidine | 50 | Inositol | Kolliphor ELP |
| 48 | 5.2 | Histidine | 50 | Maltose | Kolliphor ELP |
| 49 | 5.2 | Histidine | 10 | Maltose | Kollidon 12PF |
| 50 | 5.5 | Histidine | 50 | Maltose | Tween 40 |
| 51 | 5.5 | Histidine | 20 | Sodium chloride | Tween 40 |
| 52 | 5.8 | Histidine | 10 | Inositol | Kollidon 12PF |
| 53 | 5.8 | Histidine | 10 | Inositol | Tween 80 |
| 54 | 5.8 | Histidine | 50 | Lysine | Kolliphor ELP |
| 55 | 6 | Histidine | 50 | Sodium chloride | Tween 80 |
| 56 | 6 | Histidine | 10 | Sucrose | Kolliphor ELP |
| 57 | 6 | Histidine | 10 | Sorbitol | Tween 40 |
| 58 | 6 | Histidine | 30 | Sodium chloride | Kollidon 12PF |
| 59 | 6.5 | Histidine | 40 | Sorbitol | Kollidon 12PF |
| 60 | 6.5 | Histidine | 40 | Maltose | Tween 80 |
| 61 | 6.5 | Histidine | 50 | Sucrose | Kollidon 12PF |
| 62 | 6.5 | Histidine | 50 | Dextrose | Tween 40 |
| 63 | 6.6 | Histidine | 30 | Lysine | Tween 80 |
| 64 | 5 | Succinate | 50 | Inositol | Kollidon 12PF |
| 65 | 5 | Succinate | 10 | Maltose | Kollidon 12PF |
| 66 | 5 | Succinate | 50 | Sodium chloride | Tween 80 |
| 67 | 5.2 | Succinate | 30 | Sodium chloride | Tween 40 |
| 68 | 5.2 | Succinate | 50 | Lysine | Kolliphor ELP |
| 69 | 5.2 | Succinate | 50 | Dextrose | Kollidon 12PF |
| 70 | 5.4 | Succinate | 10 | Maltose | Tween 80 |
| 71 | 5.4 | Succinate | 30 | Inositol | Tween 40 |
| 72 | 5.4 | Succinate | 10 | Dextrose | Tween 40 |
| 73 | 5.5 | Succinate | 30 | Sodium chloride | Kollidon 12PF |
| 74 | 5.5 | Succinate | 30 | Sucrose | Kollidon 12PF |
| 75 | 5.5 | Succinate | 40 | Dextrose | Tween 80 |
| 76 | 5.8 | Succinate | 10 | Lysine | Tween 40 |
| 77 | 5.8 | Succinate | 20 | Inositol | Kolliphor ELP |
| 78 | 5.8 | Succinate | 50 | Sucrose | Kolliphor ELP |
| 79 | 6 | Succinate | 30 | Sorbitol | Tween 80 |
| 80 | 6 | Succinate | 50 | Sodium chloride | Tween 40 |

Table 4 lists the analytical tests conducted (short-term stability, mechanical stress, light exposure, F/T) in the framework of this DoE screening and presented herein.

TABLE 4
Panel of analyses conducted on DoE screening formulations

| | | Stability Study | | | |
|---|---|---|---|---|---|
| | | (4 weeks at | | | |
| | Time | 40 ± 2° C. | Light | Mechanical | Freeze/Thaw |
| Analysis | zero | 75% RH) | Stress | Stress | Stress |
| Protein content by OD | X | — | — | — | — |
| Aggregation by Optical Density | X | X | X | X | X |
| Visual Inspection | X | X | X | X | X |
| LMW Fragments by Bioanalyzer¹ | X | X | — | X | — |
| (NR) | | | | | |
| LMW and HMW by CE-SDS (NR) | X | — | X | — | — |
| HMW by SE-HPLC | X | X | X | X | X |
| Isoforms by cIEF | X | — | X | — | — |

¹2100 Bioanalyzer (Agilent)

2.1 Methods Used to Determine Stability

Thermal Stability

The thermal stability of the formulations was examined after four weeks of storage at 40±2° C. (75% R.H.) for the following:
Aggregation index: calculated by optical density to track aggregation and formation of HMW impurities
Visual inspection for presence of visual particles
HMW content by SE-HPLC (to track aggregation)
LMW content by bioanalyzer (to track fragmentation)

Light Stress

The formulations was exposed to 7 hours of light at an intensity of 765 W/m² which satisfies ICHQ1B guideline requirements. The formulations was analyzed by the following techniques:
Aggregation Index: calculated by OD, measures the extent of aggregate formation which results from light stress
Visual Inspection: for presence of visible particles resulting from aggregation
CE-SDS: for production of LMW impurities, also indicative of HMW impurities
SE-HPLC: quantitation of HMW impurities resulting from aggregation
cIEF: provides insight into relative quantity of charge variants, can monitor oxidation (by product of light stress)

Mechanical Stress

Mechanical (shaking) stress is often associated with a production of aggregates due to protein self-association and interaction among hydrophobic regions of the protein in solution. The DoE formulations in this study was examined for resistance to shaking stress after 24 hours of stirring at 200 rpm at room temperature. The shaking stress formulations was analyzed as follows:
Aggregation index: calculated by optical density to track aggregation and formation of HMW impurities
Visual inspection for presence of visual particles
HMW content by SE-HPLC (to track HMW impurity generation and hence monitor aggregations)
LMW content by bioanalyzer (to track fragmentation)

Freeze/Thaw Stress

As a protein formulation freezes, an interface is formed as micro-regions within the solution begin solidify. In these micro-environments there is a change in polarity as different component of the formulation buffer are excluded or included from the liquid matrix that is solidifying. What results is precipitation of protein as hydrophilic/hydrophobic interactions are forced upon the molecules in these changing micro-environments. To ascertain the effectiveness of the various stabilisers and surfactants in the DoE the samples were exposed to three cycles of freeze-thawing. The samples were then examined by the following analyses to determine their resistance to precipitation/aggregation/degradation by freeze-thawing:
Aggregation index: calculated by optical density to track aggregation and formation of HMW impurities
Visual inspection for presence of visual particles
HMW content by SE-HPLC (to track HMW impurity generation and hence monitor aggregations)

2.2 Manufacturing

A drug substance material of the composition: 20.6 mg/mL Avelumab, 51 mg/mL D-Mannitol, 0.6 mg/mL glacial acetic acid, pH 5.2 (surfactant-free) was equilibrated by tangential flow filtration (using a Pellicon XL Cassette Biomax cut-off 10 KDa in PES) in the three buffers:
10 mM Citrate-phosphate pH 5.2,
10 mM Glycine pH 5.2,
10 mM Histidine pH 5.2,
10 mM Succinate pH 5.2.

The buffer exchange was carried out with a 5-fold dilution of the above mentioned DS in one of the four relevant buffers and equilibrating/concentrating until the initial volume was obtained. The operation was repeated three times. The four equilibrated drug substance materials were tested for protein content by OD prior to formulations manufacturing.

Formulations 1-21 (in Citrate-Phosphate Buffer)

The exchanged DS material (26.4 mg/mL) was weighed in a glass beaker (30.30 grams). If needed, the strength of the buffer was adjusted (starting molarity of the exchanged DS: 10 mM; molarity range in the DoE formulas: 10-50 mM) by adding di-sodium hydrogen phosphate dihydrate and citric acid monohydrate. The solution was stirred until complete dissolution. The stabiliser was then added: Sorbitol (2.04 grams) or Dextrose (2.02 g) or Inositol (2.02 g) or Maltose monohydrate (4.04 g) or Lysine monohydrochloride (2.02 g) or Sodium Chloride (0.327 g) or Sucrose (3.83 g). The solution was stirred until complete dissolution. The surfactant was then added: 0.4 mL of a 50 mg/mL Tween 40 stock or 0.4 mL of a 50 mg/mL Tween 80 stock or 0.4 mL of a 50 mg/mL Kolliphor ELP stock or 20 mg of Kollidon 12PF (no stock solution needed). The solution was stirred until complete dissolution. The pH was measured and adjusted to target with diluted o-phosphoric acid or sodium hydroxide. The solution was brought to final weight (40 g) with the relevant buffer.

Formulations 22-31 (in Glycine Buffer)

The exchanged DS material (24.5 mg/mL) was weighed in a glass beaker (32.65 g). If needed, the strength of the buffer was adjusted (starting molarity of the exchanged DS: 10 mM; molarity range in the DoE formulas: 10-50 mM) by adding glycine. The solution was stirred until complete dissolution. The stabiliser was then added: Sorbitol (2.04 g) or Dextrose (2.02 g) or Inositol (2.02 g) or Maltose monohydrate (4.04 g) or Lysine monohydrochloride (2.02 g) or Sodium Chloride (0.327 g) or Sucrose (3.83 g). The solution was stirred until complete dissolution. The surfactant was then added: 0.4 mL of a 50 mg/mL Tween 40 stock or 0.4 mL of a 50 mg/mL Tween 80 stock or 0.4 mL of a 50 mg/mL Kolliphor ELP stock or 20 mg of Kollidon 12PF (no stock solution needed). The solution was stirred until complete dissolution. The pH was measured and adjusted to target with diluted hydrochloric acid or sodium hydroxide. The solution was brought to final weight (40 g) with the relevant buffer.

Formulations 32-43 (in Glycine Buffer)

The exchanged DS material (23.2 mg/mL) was weighed in a glass beaker (34.48 g). If needed, the strength of the buffer was adjusted (starting molarity of the exchanged DS: 10 mM; molarity range in the DoE formulas: 10-50 mM) by adding glycine. The solution was stirred until complete dissolution. The stabiliser was then added: Sorbitol (2.04 g) or Dextrose (2.02 g) or Inositol (2.02 g) or Maltose monohydrate (4.04 g) or Lysine monohydrochloride (2.02 g) or Sodium Chloride (0.327 g) or Sucrose (3.83 g). The solution was stirred until complete dissolution. The surfactant was then added: 0.4 mL of a 50 mg/mL Tween 40 stock or 0.4 mL of a 50 mg/mL Tween 80 stock or 0.4 mL of a 50 mg/mL Kolliphor ELP stock or 20 mg of Kollidon 12PF (no stock solution needed). The solution was stirred until complete dissolution. The pH was measured and adjusted to target with diluted hydrochloric acid or sodium hydroxide. The solution was brought to final weight (40 g) with the relevant buffer.

Formulations 64-80 (in Succinic Buffer)

The exchanged DS material (22.5 mg/mL) was weighed in a glass beaker (35.55 grams). If needed, the strength of the buffer was adjusted (starting molarity of the exchanged DS: 10 mM; molarity range in the DoE formulas: 10-50 mM) by adding succinic acid. The solution was stirred until complete dissolution. The stabiliser was then added: Sorbitol (2.04 g) or Dextrose (2.02 g) or Inositol (2.02 g) or Maltose monohydrate (4.04 g) or Lysine monohydrochloride (2.02 g) or Sodium Chloride (0.327 g) or Sucrose (3.83 g). The solution was stirred until complete dissolution. The surfactant was then added: 0.4 mL of a 50 mg/mL Tween 40 stock or 0.4 mL of a 50 mg/mL Tween 80 stock or 0.4 mL of a 50 mg/mL Kolliphor ELP stock or 20 mg of Kollidon 12PF (no stock solution needed). The solution was stirred until complete dissolution. The pH was measured and adjusted to target with diluted hydrochloric acid or sodium hydroxide. The solution was brought to final weight (40 grams) with the relevant buffer.

Formulations 44-63 (in Histidine Buffer)

The exchanged DS material (24.4 mg/mL) was weighed in a glass beaker (32.80 g). If needed, the strength of the buffer was adjusted (starting molarity of the exchanged DS: 10 mM; molarity range in the DoE formulas: 10-50 mM) by adding histidine. The solution was stirred until complete dissolution. The stabiliser was then added: Sorbitol (2.04 g) or Dextrose (2.02 g) or Inositol (2.02 g) or Maltose monohydrate (4.04 g) or Lysine monohydrochloride (2.02 g) or Sodium Chloride (0.327 g) or Sucrose (3.83 g). The solution was stirred until complete dissolution. The surfactant was then added: 0.4 mL of a 50 mg/mL Tween 40 stock or 0.4 mL of a 50 mg/mL Tween 80 stock or 0.4 mL of a 50 mg/mL Kolliphor ELP stock or 20 mg of Kollidon 12PF (no stock solution needed). The solution was stirred until complete dissolution. The pH was measured and adjusted to target with diluted hydrochloric acid or sodium hydroxide. The solution was brought to final weight (40 grams) with the relevant buffer.

Filtration and Filling

Each formulation was filtered through a 0.22 micron filter assembled on a 50 mL syringe (Millex GP 0.22 □m Express PES membrane or Millex GV 0.22 □m Durapore PVDF membrane) were used. The filtered solution was then filled in the relevant container (2 mL/container).

2.3 Results

Check of Protein Content by OD Upon Manufacturing

The protein content was determined by OD at time 0 (upon manufacturing). Values in line with the expected target (20 mg/mL) were found.

2.3.1 Thermal Stress

Aggregation Index by OD

The aggregation index was determined by OD. Additional information on aggregation index as a tool to detect sub-visible particles/larger aggregates not detectable by SE-HPLC are provided in the Annex section.

It was found that histidine buffer is generally associated to higher increases in aggregation index upon stress (i.e. larger increase in particles), most significantly when the pH is increased from 5.0 to 6.6 (pH dependent effect).

In the other buffers, changes in aggregation index are generally lower, thus indicating lower increases in sub-visible particles.

The increases in aggregation index observed in some (few) samples formulated in citrate-phosphate and glycine buffer are not directly attributable to a specific factor (e.g. stabiliser or surfactant type).

The data were statistically evaluated by ANOVA for Response Surface Linear Model, which provided the following outcome:

Statistically significant impact of buffer type, strength and pH (all have a p-value <0.001): in order to minimize the aggregation index low buffer strengths should be targeted (10 mM), in association with low pH ranges in citrate-phosphate (4.0-5.0) and glycine (4.0-5.8) and succinate (5.0-5.5), while histidine generally determines a negative impact on sub-visible particles/larger aggregates formation.

Total Aggregates by SE-HPLC

Total aggregates (HMWs) were determined by SE-HPLC at time 0 and upon thermal stress. Citrate-phosphate generally leads to higher aggregation than reference formula (reference threshold highlighted as a red horizontal bar in the chart), most particularly as pH increases. In glycine buffer, low pH ranges are to be preferred (lower than 5.0), being higher pH values associated with higher aggregation (similarly to when citrate-buffer is used). Succinate generally leads to higher aggregation values than the reference at all conditions, while histidine buffer at low pH (5.0-5.5) seems to provide aggregation values comparable to the reference.

The data were also statistically evaluated by ANOVA for Response Surface Linear Model and buffer type was confirmed to be a significant factor (p-value=0.02). Overall, in order to reduce aggregates upon thermal stress, citrate-phosphate (pH range 4.0-5.0), glycine (pH range 4.0-6.8) and histidine (pH range 5.0-5.8) should be preferred over succinate buffer.

Combinations like those present in formulations #2 (Tween 40+Dextrose in citrate-phosphate buffer pH 4.0), formulation #22 (Kollidon 12PF+Sodium chloride in glycine buffer pH 4.0) and formulation #28 (Tween 40+sodium chloride in glycine buffer pH 4.5) seem to be unfavorable to protein stabilization (significant increase in aggregation despite the optimal pH/buffer conditions applied) possibly due to incompatibility of Kollidon 12PF and Tween 40 with low pH (about 4.0-4.5)/interaction with specific stabilisers like sodium chloride.

Fragments by Bioanalyzer

Fragmentation levels were assessed by Bioanalyzer. Although no statistically significant results could be highlighted by ANOVA evaluation, conditions which were most effective in minimizing fragmentation providing LMWs percentages in line with reference composition could be highlighted:
Citrate-phosphate buffer in the pH range of 4.5-7.0
Glycine buffer in the pH range 4.0-5.8.

Considering the variability of the method (up to ±2-3% in LMWs is common when Bioanalyzer is applied), other conditions (like the remaining compositions in histidine and succinate buffers) were observed to maintain the LMWs % relatively low and are therefore worth investigating further.

Visible Particles by Visual Inspection

The presence of visible particles was assessed by visual inspection before and after thermal stress. Varying conditions in citrate-phosphate buffer can generate the presence of visible particles (most typically particulate—like suspensions) following thermal stress.

In glycine buffer, particles formation is most frequently associated to the presence of Tween species (Sample ID #23, 24, 26, 28 containing Tween 40) and formulation #30 containing Tween 80. Other formulations in glycine buffer (Sample ID from #32 to #39) showed presence of particles at time 0 which tended to decrease upon stress (possible reversible clusters).

In histidine, Tween species are generally associated to visible particles formation upon stress (all formulations showing visible particles after stress contain one of the two Tween alternatives).

In succinate buffer, particles observed at time 0 in most formulations were found to decrease upon thermal stress (possible disruption of reversible associations over time).

Summary: Thermal Stress According to SE-HPLC, OD and Bionalyzer upon thermal stress, conditions that can provide favorable performances include:
Buffers: Citrate-phosphate or glycine (preferably at more acidic pH and most relevantly in the range 4.0-5.0 for citrate phosphate and 4.0-5.8 for glycine),
Buffer strength: preferably low (as per aggregation index outcome),
Stabiliser: no specific indication obtained,
Surfactant: Kolliphor ELP observed to be effective in reducing sub-visible particles.

2.3.2 Light Stress

Aggregation Index by O.D.

Aggregation index in most DoE compositions in citrate-phosphate buffer was found to be higher than in reference formula (most significantly in the higher pH range). The pH effect was also confirmed in glycine buffer, which was however found to considerably lower the aggregation index with respect to citrate-phosphate buffer (in the pH range 4.0-4.5 values comparable with reference compositions or lower were highlighted). Histidine can generally cause considerable increases in aggregation index as well as succinate buffer (histidine remarkably worse than succinate).

The statistical analysis by ANOVA confirmed the significant impact from buffer type, pH and strength (p-value <0.0001), indicating that the best conditions to minimize particles formation include utilisation of citrate phosphate buffer (in the range 4.0-5.0 and at low buffer strength), glycine (in the range 4.0-5.8).

Surfactant was also observed to have some impact on stability, being Kolliphor ELP the best option to be taken into account when aiming at particles reduction.

Total Aggregates by SE-HPLC

Total aggregates (HMWs) were determined by SE-HPLC at time 0 and upon light stress. Citrate-phosphate generally leads to higher aggregation than reference formula, most particularly as pH increases. In glycine buffer, low pH ranges are to be preferred (lower than 4.8), being higher pH values associated with higher aggregation (similarly to when citrate-buffer is used). Succinate generally leads to higher aggregation values than the reference at all conditions, while histidine buffer (whole range aside from few exceptions) seems to provide aggregation values comparable to the reference.

The data were also statistically evaluated by ANOVA for Response Surface Linear Model and buffer type and pH were confirmed to be significant factors (p-value <0.0001).

Overall, in order to reduce aggregates upon thermal stress, glycine (pH range 4.0-5.0) and histidine (pH range 5.0-6.0) should be preferred over succinate and citrate phosphate buffers.

Importantly, stabilisers like Lysine, Dextrose, Sorbitol and Sucrose provide better stabilization against light stress than sodium chloride, maltose and Inositol (p-value <0.01).

Purity by CE-SDS

Purity as determined by CE-SDS carries the information of both HMWs and LMWs species as it is the results of the calculation: 100−% HMWs by CE-SDS−% LMWs by CE-SDS.

Purity values were determined before and after light stress.

Most formulations show higher purity than reference compositions upon light stress. Conditions that can impact negatively on stability are typically: citrate phosphate at high pH (>7.0) and glycine buffer at low pH (4.0); the latter is most probably to be explained with the negative impact from Tween 40/Kollidon 12PF at low pH.

Histidine was found to positively impact on purity, maximising formulation performances against light exposure.

Statistical analysis by ANOVA confirmed superior behaviour associated to histidine utilisation as a buffer, with comparable performances obtained when using citrate-phosphate, glycine or succinate buffers.

Isoforms Profile by cIEF

Isoforms profiles were determined at time 0 and after light exposure. Light exposure generally determines an increase in acidic isoforms due to photo-oxidation phenomena. Such increase was calculated for all DoE formulations.

Several conditions are favourable to protein stabilization (i.e. lower changes in isoforms profile), such as citrate-phosphate and glycine buffer (most typically in the lower pH range). Lower performances observed when histidine is used as formulation buffer. The data, evaluated by ANOVA for Response Surface Linear Model confirmed the above (buffer type statistically significant factor with p-value <0.0001).

The statistical analysis also confirmed a positive impact (reduction in acidic isoforms change) when L-Lysine is used as stabiliser. The effect is quite clear when observing the changes found in formulations #11, 29, 31, 38, considerably lower than those in the surrounding formulation space with alternative stabilisers.

Visible Particles by Visual Inspection

The presence of visible particles was assessed by visual inspection before and after light stress. Most formulations are not impacted by light stress in terms of visible particles. No specific conditions related to particle formation upon light stress.

Summary: Light Exposure Stress

According to SE-HPLC, OD, CE-SDS, cIEF and visual inspection upon light stress, conditions that can provide favorable performances include:
Buffers: glycine buffer (preferably at more acidic pH and most relevantly in the range 4.0-4.5),
Buffer strength: preferably low (as per aggregation index outcome),
Stabiliser: Lysine (monohydrochloride), dextrose and sorbitol showed a positive impact on protein stability
Surfactant: Kolliphor ELP observed to be effective in reducing sub-visible particles

2.3.3 Freeze-Thawing

Aggregation Index by Optical Density

After 3× freeze-thawing cycles (−80° C.→room temperature), once again, glycine buffer (low pH) is confirmed to provide the lowest values indicating lower particle formation. An increase in aggregation index is observed both in citrate-phosphate buffer and glycine buffer as pH increase (pH effect more critical in citrate-phosphate buffer). Generally higher aggregation index values than reference composition are observed in histidine and succinate buffers.

The statistical analysis by ANOVA highlighted a moderately significant impact from buffer type, pH and surfactant type (0.01<p-value <0.05), indicating that citrate-phosphate and glycine buffers at pH lower than 6.0 are the best option for protein stabilisation against particles formation induced by freeze-thawing, being succinate and histidine buffer slightly pejorative with respect to reference composition.

A comparison of the impact of the different surfactants shows comparable performances from Tween 80, Kollidon 12PF and Kolliphor ELP (slightly preferable), while Tween 40 is expected to increase aggregation index.

Total Aggregates by SE-HPLC

All formulations show lower total aggregates than reference composition upon freeze-thawing stress (values comparable to time 0).

In citrate-phosphate buffer, aggregates tend to increase up to the level of reference composition as the primary effect of pH (2.0-2.5% HMWs) being increased up to the range 7.0-7.5 with minor/negligible changes upon freeze-thawing, whilst at pH <7.0 total aggregates typically amount to lower than 1.5% (before and after stress). In glycine and histidine buffer all total aggregates values after stress amount to less than 1% (comparable with time 0 values). In succinate, freeze-thawing was not found to determine critical changes with respect to time 0, however total aggregates are generally slightly higher than in glycine and histidine (still equal to or lower than 1.5%, i.e. considerably lower than reference after stress).

Statistical analysis confirmed the significant impact from buffer type and pH (p-value <0.0001), being citrate-phosphate buffer (pH 4.0-6.0), glycine buffer (pH 4.0-7.0) and histidine (5.0-6.6) the best options for protein stabilisation against freeze-thawing.

A significant impact (p-value <0.01) was also highlighted for the stabiliser type factor: Lysine hydrochloride minimises time 0 aggregation and the effects related to freeze-thawing stress (cf. Sample ID #6-9-11-17 in citrate-buffer); sucrose and dextrose, similarly, show stabilising properties.

Visible Particles by Visual Inspection

In the results of visual inspection upon freeze-thawing the general trends that can be highlighted:
In citrate-phosphate, particle formation is more likely at higher pH,
In glycine buffer at low pH (<5), particle formation is primarily related to the presence of Tween 40 (destabilising surfactant),
In histidine buffer, Tween species are generally related to particle formation,
In succinate, no specific factors seem to be related to particle formation, which is however quite a frequent occurrence when this buffer is used.

Summary: Freeze-Thawing Stress

According to SE-HPLC, OD and visual inspection upon 3× freeze-thawing cycles (−80° C.→room temperature), conditions that can provide favorable improved performances include:
Buffers: glycine or citrate-phosphate buffers (preferably at more acidic pH and most relevantly in the range 4.0-6.0),
Stabiliser: Lysine (monohydrochloride), dextrose and sucrose showed a positive impact on protein stability (reduction of total aggregates by SE-HPLC),
Surfactant: incompatibilities of Tween species with glycine and histidine buffered formulations are to be taken into account and avoided to minimize visible particles formation.

2.3.4 Mechanical Stress

Aggregation Index by Optical Density

As previously shown, the factors that allow aggregation index values most similar to reference (i.e. minimal or no increases with respect to time 0) are: Citrate-phosphate generally leads to higher aggregation index values than reference, most particularly as pH increases and in presence of Tween species: Sample ID #2

(Tween 40), #8 (Tween 80), #11 (Tween 40), #19 (Tween 40), #21 (Tween 40). Glycine provides a conspicuous stabilising effect in the low pH range (aggregation index values slightly lower than reference).

Histidine buffer is to be preferably used at pH values close to 5.0 and without Tween 40 and Tween 80, which appear to be related to the highest aggregation index values: Sample ID #50 (Tween 40), #60 (Tween 80), #62 (Tween 40).

Succinate generally leads to aggregation index values slightly higher than reference composition, regardless of the specific factors involved.

The above results were confirmed by ANOV, which indicated buffer type and pH as statistically significant factors (p-value <0.01) and surfactant as moderately significant factor (0.01<p-value <0.05).

Glycine buffer at low pH (4.0-5.5) is highlighted as the selection buffer to minimise the aggregation index. The tendency towards an increase in aggregation index given by Tween species (Tween 40 worse than Tween 80) is confirmed by the surface response models.

Total Aggregates by SE-HPLC

Minimal increase with respect to time 0 were observed for most formulations indicating a minor impact from this type of stress. Differentiation in terms of total aggregates appears to be the primary effect of buffer type and pH, as already highlighted Buffer type and pH confirmed to be statistically significant factors by ANOVA (p-value <0.0001); as well as buffer strength (p-value <0.01) and stabiliser type (0.01<p-value <0.05).

Preferable ranges and conditions to minimise aggregates to the level of reference composition (<1%) include: citrate-phosphate buffer (pH <5 and low ionic strength); glycine buffer (whole pH and ionic strength range); histidine buffer (whole range) and succinate buffer (pH 5.0-5.5 and low ionic strength). Preferable stabilisers are L-Lysine monohydrochloride, Maltose, Sucrose and Dextrose.

Fragments by Bioanalyzer

Except for Sample ID #22-23-24 (in glycine buffer, pH 4.0, containing Tween 40 or Kollidon 12PF), the remaining formulations showed LMWs % comparable to or lower than reference composition upon mechanical stress, also taking into account the variability of this method (±2-3% in LMWs % results is characteristic). Therefore, it can be concluded that most conditions tested can help improve protein resistance against fragmentation provided that combinations like glycine buffer (low pH)+Tween 40 are avoided.

The statistical elaboration highlighted the better performances of formulations in succinate and histidine buffers, to be however carefully considered and evaluated as substantially comparable to/slightly better than the other formulas in citrate-phosphate and glycine buffer due to the above discussed method variability.

Visible Particles by Visual Inspection

In the results of visual inspection upon freeze-thawing are the general trends that can be highlighted:
In citrate-phosphate buffer (Sample ID #1-21), particle formation occurs at almost all conditions regardless of specific factors involved,
In glycine buffer, particle formation is primarily related to the presence of Tween 40 (Sample ID #23, 26, 28 and Kollidon 12PF (Formulations #22, 32, 37, 43)
In histidine buffer, all formulas showing increase of visible particles upon mechanical shaking contain either Tween 40 or Tween 80,
In succinate, no specific factors seem to be related to particle formation.

Summary: Mechanical Stress

According to SE-HPLC, OD, Bioanalyzer and visual inspection upon mechanical shaking, conditions that can provide favorable performances with respect to reference compositions include:
Buffers: glycine (preferably at more acidic pH and most relevantly in the range 4.0-5.5), histidine and succinate at pH of about 5.0.
Stabiliser: Lysine (monohydrochloride), Sucrose, Maltose and Dextrose showed a positive impact on protein stability (reduction of total aggregates by SE-HPLC),
Surfactant: incompatibilities of Tween species with glycine, citrate-phosphate and histidine buffered formulations are to be taken into account and avoided to minimize visible particles formation.

### Example 3—Formulations Optimisation

3.1 Formulation Optimisation

The data shown in Example 2 were combined to identify the formulation space which could suitably stabilise Avelumab (factors evaluated: buffer type, pH and strength, stabiliser type and surfactant) against thermal, freeze-thaw, mechanical and light stress.

Using the following criteria
Minimise HMWs (by SE-HPLC) after thermal stress, mechanical shaking, freeze-thawing and light stress,
Minimise LMWs (by Bioanalyser) after thermal stress and mechanical shaking,
Maximise purity (by CE-SDS) after light stress,
Minimise acidic isoforms (by cIEF) change after light stress,
Target Aggregation index values (by OD) lower than 2 after thermal stress, mechanical shaking, freeze-thawing and light stress, for each buffer type the 10 most promising formulations were extrapolated as shown in Table 5.

TABLE 5
Candidate formulations (DoE extrapolation)

| | Buffer | Buffer strength | Surfactant | Stabiliser | Buffer |
|---|---|---|---|---|---|
| Number | pH | (mM) | (0.5 mg/mL) | (280 mM) | type |
| 1 | 4.4 | 10 | Kolliphor ELP | Lysine | Glycine |
| 2 | 4.1 | 10 | Tween 80 | Lysine | Glycine |
| 3 | 4.0 | 10 | Tween 80 | Lysine | Glycine |
| 4 | 4.0 | 10 | Kolliphor ELP | Dextrose | Glycine |
| 5 | 4.0 | 10 | Kolliphor ELP | Dextrose | Glycine |
| 6 | 4.0 | 10 | Kolliphor ELP | Dextrose | Glycine |
| 7 | 4.0 | 10 | Kollidon 12PF | Lysine | Glycine |
| 8 | 4.0 | 10 | Kolliphor ELP | Sorbitol | Glycine |
| 9 | 4.0 | 10 | Kolliphor ELP | Sucrose | Glycine |
| 10 | 4.0 | 10 | Kolliphor ELP | Sucrose | Glycine |
| 1 | 4.0 | 10 | Kollidon 12PF | Sorbitol | Citrate-phosphate |
| 2 | 4.2 | 15 | Kollidon 12PF | Lysine | Citrate-phosphate |
| 3 | 4.3 | 17 | Kollidon 12PF | Sucrose | Citrate-phosphate |
| 4 | 4.1 | 20 | Kollidon 12PF | Lysine | Citrate-phosphate |
| 5 | 4.1 | 15 | Tween 80 | Lysine | Citrate-phosphate |
| 6 | 4.0 | 27 | Kollidon 12PF | Sucrose | Citrate-phosphate |
| 7 | 4.1 | 19 | Kolliphor ELP | Sucrose | Citrate-phosphate |
| 8 | 4.1 | 22 | Kolliphor ELP | Dextrose | Citrate-phosphate |
| 9 | 4.2 | 13 | Tween 80 | Sorbitol | Citrate-phosphate |
| 10 | 4.2 | 17 | Kolliphor ELP | Dextrose | Citrate-phosphate |
| 1 | 5.0 | 10 | Kolliphor ELP | Dextrose | Histidine |
| 2 | 5.0 | 10 | Kolliphor ELP | Dextrose | Histidine |
| 3 | 5.0 | 10 | Kolliphor ELP | Sorbitol | Histidine |
| 4 | 5.0 | 10 | Kolliphor ELP | Sucrose | Histidine |
| 5 | 5.0 | 11 | Kolliphor ELP | Sucrose | Histidine |
| 6 | 5.1 | 10 | Kolliphor ELP | Sorbitol | Histidine |
| 7 | 5.1 | 10 | Kolliphor ELP | Sucrose | Histidine |
| 8 | 5.0 | 10 | Kolliphor ELP | Inositol | Histidine |
| 9 | 5.0 | 15 | Kolliphor ELP | Sorbitol | Histidine |
| 10 | 5.0 | 10 | Kolliphor ELP | Lysine | Histidine |
| 1 | 5.0 | 10 | Kollidon 12PF | Sucrose | Succinate |
| 2 | 5.0 | 10 | Kolliphor ELP | Lysine | Succinate |
| 3 | 5.0 | 10 | Kolliphor ELP | Lysine | Succinate |
| 4 | 5.0 | 12 | Kolliphor ELP | Lysine | Succinate |
| 5 | 5.1 | 10 | Kolliphor ELP | Lysine | Succinate |
| 6 | 5.1 | 10 | Kollidon 12PF | Sucrose | Succinate |
| 7 | 5.0 | 10 | Kollidon 12PF | Sorbitol | Succinate |
| 8 | 5.0 | 10 | Kollidon 12PF | Lysine | Succinate |
| 9 | 5.0 | 10 | Kollidon 12PF | Dextrose | Succinate |
| 10 | 5.0 | 14 | Kollidon 12PF | Sucrose | Succinate |

3.2 Lead Formulations to be Further Assessed

Out of the formulations of Table 5, the eleven formulations listed in Table 6 appeared most promising. Hence, they were manufactured and evaluated upon thermal stress and repeated freeze-thawing cycles as per the analytical panel shown in Table 7.

Thermal stress was selected as the most relevant stress conditions to evaluate formulation performances and possibly predict stability at refrigerated conditions. Freeze-thawing was also considered in order to anticipate any issues related to temperature excursions/storage of pre-formulated DS materials.

The results of the experiments carried out on these formulation are described in the following paragraphs.

TABLE 6
Lead formulations resulting from DoE

| | | | Buffer | | Surfactant |
|---|---|---|---|---|---|
| | pH | | Strength | | (0.5 |
| DP | (±0.1 | Buffer | (mM) | Stabiliser | mg/mL) |
| 1 | 4.4 | Glycine | 10 | Lysine | Kolliphor ELP |
| | | | | (monohydrochloride) | |
| | | | | 280 mM | |
| 2 | 4.4 | Glycine | 10 | Lysine | Kolliphor ELP |
| | | | | (monohydrochloride) | |
| | | | | 140 mM | |
| 3 | 4.4 | Glycine | 10 | Lysine (monohydrate) | Kolliphor ELP |
| | | | | 280 mM | |
| 4 | 4.4 | Glycine | 10 | Lysine acetate | Kolliphor ELP |
| | | | | 140 mM | |
| 5 | 4.1 | Glycine | 10 | Lysine (monohydrate) | Tween 80 |
| | | | | 280 mM | |
| 6 | 5.0 | Histidine | 10 | Dextrose | Kolliphor ELP |
| | | | | 280 mM | |
| 7 | 5.0 | Histidine | 10 | Sucrose | Kolliphor ELP |
| | | | | 280 mM | |
| 8 | 4.2 | Citrate- | 15 | Lysine | Kollidon 17PF |
| | | Phosphate | | (monohydrochloride) | |
| | | | | 140 mM | |
| 9 | 4.3 | Citrate- | 17 | Sucrose | Kollidon 17PF |
| | | Phosphate | | 280 mM | |
| 10 | 5.0 | Succinate | 10 | Lysine | Kolliphor ELP |
| | | | | (monohydrochloride) | |
| | | | | 140 mM | |
| 11 | 5.0 | Succinate | 10 | Sucrose | Kollidon 17PF |
| | | | | 280 mM | |

TABLE 7
Panel of analyses conducted on lead formulations

| | | Thermal Stress | Freeze- |
|---|---|---|---|
| | Time | (4 weeks at 40 ± | thaw |
| Test | 0 | 2° C. 75% RH) | 3X |
| Visible particles (visual) | X | X | X |
| pH | X | X | — |
| Turbidity (OD) | X | X | X |
| Sub-visible particles (PAMAS) | X | X | X |
| Protein content (OD) | X | X | — |
| HMWs by SE-HPLC | X | X | X |
| LMWs by Bioanalyzer | X | X | — |
| Isoforms profile by iCE | X | X | — |
| Tertiary structure by CD | X | X | — |

3.3 Manufacturing of Lead Formulations Resulting from DoE Step

A drug substance material of the composition: 18.6 mg/mL avelumab, 51 mg/mL D-Mannitol, 0.6 mg/mL glacial acetic acid, pH 5.2 (surfactant-free) was equilibrated by tangential flow filtration (using a Pellicon XL Cassette Biomax cut-off 50 KDa in PES) in the three buffers:

### 10 mM Glycine pH 4.4,

10 mM histidine pH 5.0,
15 mM citrate-phosphate pH 4.2,
10 mM succinate pH 5.0.

The buffer exchange was carried out with a 5-fold dilution of the above mentioned DS in one of the four relevant buffers and equilibrating/concentrating until the initial volume was obtained. The operation was repeated three times. The four equilibrated drug substance materials were tested for protein content by OD prior to formulations manufacturing.

Formulations 1-5 (in Glycine Buffer)

The exchanged DS material (21.8 mg/mL) was weighed in a glass beaker (64.2 g). The stabiliser was then added: Lysine monohydrochloride (3.58 grams for DP1 or 1.79 g for DP2) or Lysine monohydrate (3.22 grams for DP3 and DP5) or Lysine Acetate (2.02 g for DP4). The solution was stirred until complete dissolution. The surfactant was then added: 0.7 mL of a 50 mg/mL Kolliphor ELP stock (in 10 mM glycine pH 4.4 for DP 1-2-3-4) or 0.7 mL of a 50 mg/mL Tween 80 (in 10 mM glycine pH 4.1 for DP5). The solution was stirred until complete dissolution. The pH was measured and adjusted to target with diluted hydrochloric acid or sodium hydroxide. The solution was brought to final weight (70 g) with the relevant buffer.

Formulations 6-7 (in Histidine Buffer)

The exchanged DS material (23.2 mg/mL) was weighed in a glass beaker (60.3 g). The stabiliser was then added: Dextrose (3.53 g for DP6) or Sucrose (6.71 g for DP7). The solution was stirred until complete dissolution. The surfactant was then added: 0.7 mL of a 50 mg/mL Kolliphor ELP stock (in 10 mM histidine buffer pH 5.0 for DP6 and 7). The solution was stirred until complete dissolution. The pH was measured and adjusted to target (pH 5.0) with diluted hydrochloric acid or sodium hydroxide. The solution was brought to final weight (70 g) with relevant buffer (10 mM histidine buffer pH 5.0).

Formulations 8-9 (in Citrate-Phosphate Buffer)

The exchanged DS material (23.4 mg/mL) was weighed in a glass beaker (59.8 g). If needed (DP9), the strength of the buffer was adjusted by adding citric acid (monohydrate) and di-sodium phosphate hydrogen (dihydrate). The stabiliser was then added: Lysine monohydrochloride (1.79 g for DP8) or Sucrose (6.71 g for DP9). The solution was stirred until complete dissolution. The surfactant was then added: 35 mg of Kollidon 17PF (for both DP8 and 9). The solution was stirred until complete dissolution. The pH was measured and adjusted to target (pH 4.2 for DP8 and 4.3 for DP9) with diluted o-phosphoric acid or sodium hydroxide. The solution was brought to final weight (70 g) with the relevant buffer.

Formulations 10-11 (in Succinate Buffer)

The exchanged DS material (24.5 mg/mL) was weighed in a glass beaker (57.1 gra g ms). The stabiliser was then added: Lysine monohydrochloride (1.79 g for DP10) or Sucrose (6.71 g for DP11). The solution was stirred until complete dissolution. The surfactant was then added: 0.7 mL of a 50 mg/mL Kolliphor ELP stock solution in 10 mM succinate buffer pH 5.0 (DP10) or 35 mg of Kollidon 17PF (DP11). The solution was stirred until complete dissolution. The pH was measured and adjusted to target (pH 5.0 for DP10 and 11) with diluted hydrochloric acid or sodium hydroxide. The solution was brought to final weight (70 g) with 10 mM succinate buffer pH 5.0.

3.4 Results

3.4.1 Thermal Stress

Protein Content by OD:

No major changes observed with respect to time 0 after 4 weeks at 40° C.

pH:

The pH values at time 0 were in line with the target. No major changes were observed with respect to time 0 after 4 weeks at 40° C.

Visible Particles by Visual Inspection

All formulations were found to be free of visible particles at time 0. Upon stress, one formulation (DP6) showed the presence of particles (possibly formulation-related).

Turbidity by Nephelometry

Most formulations have turbidity values in the clear or slightly opalescent range with minimal changes after stress (DP 2-4-6-7-9-10-11). Other formulations show either higher turbidity changes from the slightly opalescent to the opalescent range (DP1) or values in the opalescent range already at time 0 with minor/negligible changes after stress (DP 3-8). Formulation DP5 shows a significant increase in turbidity (>18 NTU) after stress.

Sub-Visible Particles by Light Obscuration

Particles ≥25 micron were well below the Pharmacopoeia limit of 600 particles/container (typically <100 particles).

Particles ≥10 micron had somewhat larger counts, but were still below the 6000 particles/container limit. DP8 and 9, in citrate-phosphate buffer, showed higher counts than the others (still below the above limit) at time 0, with significant reduction after stress.

Total Aggregates by SE-HPLC

With respect to total aggregates by SE-HPLC at time 0 and after thermal stress, DP 1-2-3-4 (glycine buffer) varied for the stabiliser type and amount, but had the same buffer strength, surfactant and pH): reduction in Lysine monohydrochloride from 280 mM (DP1) to 140 mM (DP2) seems to favor protein stability. The higher aggregation rate was confirmed when Lysine monohydrate at 280 mM was used (DP3). Lysine acetate (140 mM) provided similar performances as Lysine monohydrochloride used at the same concentration (DP2).

DP5 (glycine buffer) showed significant increase in aggregates (probably due an unfavourable combination of Lysine monohydrate at 280 mM+Tween 80 instead of Kolliphor ELP).

DP6-7 (histidine buffer) showed no changes in aggregates.

DP8-9 (citrate-phosphate buffer): sucrose in DP9 seems to be the critical factor which can significantly improve formulation performance with respect to DP8 (Lysine monohydrate) being the other ingredients/parameters pretty similar (same buffer type, same surfactant and similar pH: 4.2 vs. 4.3).

DP10-11 (succinate buffer): no significant changes in aggregation were observed (similar performances of Lysine monohydrate and Sucrose in this buffer).

Lower Molecular Weights by Bioanalyzer

Fragments by Bioanalyzer at time 0 and after thermal stress:

DP 1-2-3-4 (glycine buffer) varied for the stabiliser type and amount, but had the same buffer strength, surfactant and pH): similar increase in fragments (+3-5% after stress).

DP5 (glycine buffer) showed significant increase in lower molecular weight species (probably due an unfavourable combination of Lysine monohydrate at 280 mM+Tween 80 instead of Kolliphor ELP): +13% increase after stress.

DP6-7 (histidine buffer) showed no changes in fragments.

DP8-9 (citrate-phosphate buffer): sucrose in DP9 (+6% in fragments after stress) seems to be the critical factor which can significantly improve formulation performance with respect to DP8 (Lysine monohydrate; +11% in fragments) being the other ingredients/parameters pretty similar (same buffer type, same surfactant and similar pH: 4.2 vs. 4.3).

DP10-11 (succinate buffer): minimal changes for both (similar performances of Lysine monohydrate and Sucrose in this buffer): +1-3% in lower molecular weight species after stress.

Isoforms Profile by cIEF

Isoforms profile at time 0 and after thermal stress: Upon thermal stress all samples generally tended to lose part of the main species with concurrent increase in acidic species and minor changes in the basic isoforms. More in detail: DP 1-2-3-4-5 (glycine buffer): similar changes were observed in isoforms profile. For the five samples, main species decreased by about 10-12% (increase in acidic isoforms of 14-17% and decrease in basic isoforms of −4/−6%).

DP 6-7 (histidine buffer): DP6 showed major changes in isoforms profile and the profiles obtained could not be elaborated due to likely instability from the components chosen and/or contamination of the sample prior to analysis. DP7 showed changes similar to samples in glycine buffer.

DP8-9 (citrate-phosphate buffer): significant changes in both formulations, higher than observed in the other buffers. Acidic species were found to increase up to 24-29% after stress.

DP10-11 (succinate buffer): DP10 showed minimal changes, even lower than the other samples in the other buffers: main species decreased by about 7% (increase in acidic isoforms of about 12% and decrease in basic isoforms of about −5%). DP11 showed higher changes (increase in acidic isoforms after stress was +20%).

Tertiary Structure by Circular Dichroism

Circular dichroism was run before and after stress on the lead formulations.

The samples were diluted with WFI to 1.5 mg/mL and then tested in 1 cm—pathlength quartz cuvettes with a Jasco J-810 spectropolarimeter in the range 250 nm-320 nm at a scanning speed of 20 nm/min (sensitivity: standard; bandwidth: 1 mm; data pitch 0.2 nm; D.I.T.: 8 seconds; 4 replicates) at room temperature.

Protein conformation in most formulations could be effectively retained, with only slight changes in the region 260-280 nm (tyrosine and phenyalanine signals). However, a few exceptions could be observed, where more significant changes could be found which may indicate partial disruption/unfolding and loss of structure following thermal stress: DP5 (possible effect of the surfactant type present), DP8 and 9 (formulations in citrate-phosphate buffer; possible effect of the buffer type and combination with other ingredients present).

3.4.2 Freeze-Thawing

Visible Particles by Visual Inspection

Repeated FT cycles were not observed to cause significant increase in visible particles. Some formulations presented fibers-like particles upon stress (not particulate/precipitate or other forms typically formulation-related).

Turbidity by Nephelometry

Upon freeze-thawing, no significant changes occur in the formulations tested. Most formulations are clear or slightly opalescent at time 0 and after stress (exception: DP3, 5, 8, opalescent solution range at time 0, with negligible changes after stress).

Sub-Visible Particles by Light Obscuration Method

Particles ≥25 micron were well below the Pharmacopoeia limit of 600 particles/container (typically ≤100 particles).

Particles ≥10 micron had larger counts, but still below the 6000 particles/container limit. DP8 and 9, in citrate-phosphate buffer, show higher counts than the others (still below the above limit) at time 0, with no further increase upon FT stress.

Total Aggregates by SE-HPLC

In the total aggregates by SE-HPLC before and after FT stress, minimal changes were observed for all formulations (total aggregates increased by 0.2-0.5% after 3 FT cycles).

3.5 Conclusion

In glycine buffer, the most suitable conditions for antibody stabilisation include:

low ionic strength (10 mM),
low pH (4.0-4.4),

Lysine (monohydrochloride), Dextrose, Sucrose and Sorbitol as stabilisers, Preferred surfactants: Kolliphor ELP and Kollidon 12PF (Tween 80 to be possibly avoided to due visible particles concerns).

In succinate buffer, the most suitable conditions for antibody stabilisation include:

low ionic strength (10 mM),
pH 5.0-5.1

Lysine (monohydrochloride), Dextrose, Sucrose or Sorbitol as stabilisers, Preferred surfactants: Kolliphor ELP and Kollidon 12PF (Tween 80 to be possibly avoided to due visible particles concerns).

In citrate-phosphate buffer, the most suitable conditions for antibody stabilisation include:

low ionic strength (10-30 mM),
low pH (4.0-4.5),

Lysine (monohydrochloride), Dextrose, Sucrose or Sorbitol as stabilisers,

Preferred surfactants: Kolliphor ELP and Kollidon 12PF (Tween 80 to be possibly avoided to due visible particles concerns).

In histidine buffer, the most suitable conditions for antibody stabilisation include:

low ionic strength (10-15 mM),
pH 5.0-5.1,

Dextrose, Sucrose, Lysine (monohydrochloride), Inositol, Sorbitol as stabilisers, Preferred surfactants: Kolliphor ELP and Kollidon 12PF (Tween 80 to be possibly avoided to due visible particles concerns).

The most favourable formulations of Table 6 were found to be DP 2, 4, 7, and 10.

1. An aqueous pharmaceutical antibody formulation, comprising:
(i) Avelumab in a concentration of 1 mg/mL to 30 mg/mL as the antibody;
(ii) glycine, succinate, citrate phosphate or histidine in a concentration of 5 mM to 35 mM as the buffering agent;
(iii) lysine monohydrochloride, lysine monohydrate, lysine acetate, dextrose, sucrose, sorbitol or inositol in a concentration of 100 mM to 320 mM as the stabiliser;
(iv) povidone, polyoxyl castor oil or polysorbate in a concentration of 0.25 mg/mL to 0.75 mg/mL, as the surfactant;
wherein the formulation does not comprise methionine, and
further wherein the formulation has a pH of 3.8 to 5.2.

2. The formulation of claim 1, wherein the formulation does not comprise an antioxidant.

3. The formulation of claim 1, wherein the concentration of Avelumab is about 10 mg/mL to about 20 mg/mL.

4. The formulation of claim 1-3, wherein the concentration of said glycine, succinate, citrate phosphate or histidine is about 10 mM to about 20 mM.

5. The formulation of claim 1-3, wherein the concentration of said lysine monochloride is about 140 mM to about 280 mM, or the concentration of said lysine monohydrate is about 280 mM, or the concentration of the said lysine acetate is about 140 mM.

6. The formulation of claim 1-3, wherein the concentration of said dextrose, sucrose, sorbitol or inositol is about 280 mM.

7. The formulation of claim 1-3, wherein the concentration of said povidone, polyoxyl castor oil or polysorbate is about 0.5 mg/mL.

8. The formulation of claim 1-3, wherein the said povidone is the low molecular weight povidone Kollidon 12PF or 17PF, or wherein the said polyoxyl castor oil is Polyoxyl 35 Castor Oil, or wherein the said polysorbate is Polysorbate 80.

9. The formulation of any one of claims 1-8, wherein the concentration of Avelumab is about 20 mg/ml.

10. The formulation of claim 2, comprising
(i) Avelumab in a concentration of 1 mg/mL to about 20 mg/mL as the antibody;
(ii) glycine in a concentration of 5 mM to 15 mM as the buffering agent, and not comprising any other buffering agent;
(iii) lysine monohydrochloride, dextrose, sucrose or sorbitol in a concentration of 100 mM to 320 mM as the stabiliser, and not comprising any other stabiliser;
(iv) Kollidon 12PF, polyoxyl 35 castor oil or Polysorbate 80 in a concentration of 0.25 mg/mL to 0.75 mg/mL, as the surfactant, and not comprising any other surfactant;
wherein the formulation has a pH of 3.8 to 4.6.

11. The formulation of claim 2, comprising
(i) Avelumab in a concentration of 1 mg/mL to about 20 mg/mL as the antibody;
(ii) succinate in a concentration of 5 mM to 15 mM as the buffering agent, and not comprising any other buffering agent;
(iii) lysine monohydrochloride, dextrose, sucrose or sorbitol in a concentration of 100 mM to 320 mM as the stabiliser, and not comprising any other stabiliser;
(iv) Kollidon 12PF or polyoxyl 35 castor oil in a concentration of 0.25 mg/mL to 0.75 mg/mL, as the surfactant, and not comprising any other surfactant;
wherein the formulation has a pH of 4.9 to 5.2.

12. The formulation of claim 2, comprising
(i) Avelumab in a concentration of 1 mg/mL to about 20 mg/mL as the antibody;
(ii) citrate phosphate in a concentration of 10 mM to 20 mM as the buffering agent, and not comprising any other buffering agent;
(iii) lysine monohydrochloride, dextrose, sucrose or sorbitol in a concentration of 100 mM to 320 mM as the stabiliser, and not comprising any other stabiliser;
(iv) Kollidon 12PF or polyoxyl 35 castor oil in a concentration of 0.25 mg/mL to 0.75 mg/mL, as the surfactant, and not comprising any other surfactant;
wherein the formulation has a pH of 3.8 to 4.7.

13. The formulation of claim 2, comprising
(i) Avelumab in a concentration of 1 mg/mL to about 20 mg/mL as the antibody;
(ii) histidine in a concentration of 5 mM to 15 mM as the buffering agent, and not comprising any other buffering agent;
(iii) lysine monohydrochloride, dextrose, sucrose, inositol or sorbitol in a concentration of 100 mM to 320 mM as the stabiliser, and not comprising any other stabiliser;
(iv) Kollidon 12PF or polyoxyl 35 castor oil in a concentration of 0.25 mg/mL to 0.75 mg/mL, as the surfactant, and not comprising any other surfactant;
wherein the formulation has a pH of 4.8 to 5.2.

14. The formulation of claim 10, comprising
(i) Avelumab in a concentration of 1 mg/mL to about 20 mg/mL as the antibody;
(ii) glycine in a concentration of about 10 mM as the buffering agent, and not comprising any other buffering agent;
(iii) lysine monohydrochloride in a concentration of about 140 mM as the stabiliser, and not comprising any other stabiliser;
(iv) polyoxyl 35 castor oil in a concentration of about 0.5 mg/mL as the surfactant, and not comprising any other surfactant;
wherein the formulation has a pH of 4.2 to 4.6.

15. The formulation of claim 10, comprising
(i) Avelumab in a concentration of 1 mg/mL to about 20 mg/mL as the antibody;
(ii) glycine in a concentration of about 10 mM as the buffering agent, and not comprising any other buffering agent;
(iii) lysine acetate in a concentration of about 140 mM as the stabiliser, and not comprising any other stabiliser;
(iv) polyoxyl 35 castor oil in a concentration of about 0.5 mg/mL as the surfactant, and not comprising any other surfactant;
wherein the formulation has a pH of 4.2 to 4.6.

16. The formulation of claim 13, comprising
(i) Avelumab in a concentration of 1 mg/mL to about 20 mg/mL as the antibody;
(ii) histidine in a concentration of about 10 mM as the buffering agent, and not comprising any other buffering agent;
(iii) sucrose in a concentration of about 280 mM as the stabiliser, and not comprising any other stabiliser;
(iv) Kollidon 12PF in a concentration of about 0.5 mg/mL as the surfactant, and not comprising any other surfactant;
wherein the formulation has a pH of 4.8 to 5.2.

17. The formulation of claim 11, comprising
(i) Avelumab in a concentration of 1 mg/mL to about 20 mg/mL as the antibody;
(ii) succinate in a concentration of about 10 mM as the buffering agent, and not comprising any other buffering agent;
(iii) lysine monohydrochloride in a concentration of about 140 mM as the stabiliser, and not comprising any other stabiliser;
(iv) polyoxyl 35 castor oil in a concentration of about 0.5 mg/mL as the surfactant, and not comprising any other surfactant;
wherein the formulation has a pH of 4.8 to 5.2.

18. The formulation of claim 14, consisting of:
(i) Avelumab in a concentration of 20 mg/mL;
(ii) glycine in a concentration of 10 mM;
(iii) lysine monohydrochloride in a concentration of 140 mM;
(iv) polyoxyl 35 castor oil in a concentration of 0.5 mg/mL;
(v) HCl of NaOH to adjust the pH;
(vi) water (for injection) as the solvent;
wherein the formulation has a pH of 4.4 (±0.1).

19. The formulation of claim 15, consisting of:
(i) Avelumab in a concentration of 20 mg/mL;
(ii) glycine in a concentration of 10 mM;
(iii) lysine acetate in a concentration of 140 mM;
(iv) polyoxyl 35 castor oil in a concentration of 0.5 mg/mL;
(v) HCl of NaOH to adjust the pH;
(vi) water (for injection) as the solvent;
wherein the formulation has a pH of 4.4 (±0.1).

20. The formulation of claim 16, consisting of:
(i) Avelumab in a concentration of 20 mg/mL;
(ii) histidine in a concentration of 10 mM;
(iii) sucrose in a concentration of 280 mM;
(iv) Kollidon 12PF in a concentration of 0.5 mg/mL;
(v) HCl of NaOH to adjust the pH;
(vi) water (for injection) as the solvent;
wherein the formulation has a pH of 5.0 (±0.1).

21. The formulation of claim 17, consisting of:
(i) Avelumab in a concentration of 20 mg/mL;
(ii) succinate in a concentration of 10 mM;
(iii) lysine monohydrochloride in a concentration of 140 mM;
(iv) polyoxyl 35 castor oil in a concentration of 0.5 mg/mL;
(v) HCl of NaOH to adjust the pH;
(vi) water (for injection) as the solvent;
wherein the formulation has a pH of 5.0 (±0.1).

22. The formulation of any of claims 1-21, wherein said Avelumab has the heavy chain sequence of either (SEQ ID NO:1) or (SEQ ID NO:2), the light chain sequence of (SEQ ID NO:3), and carries a glycosylation on Asn300 comprising FA2 and FA2G1 as the main glycan species, having a joint share of >70% of all glycan species.

23. The formulation of claim 22, wherein in the Avelumab glycosylation said FA2 has a share of 44%-54% and said FA2G1 has a share of 25%-41% of all glycan species.

24. The formulation of claim 23, wherein in the Avelumab glycosylation said FA2 has a share of 47%-52% and said FA2G1 has a share of 29%-37% of all glycan species.

25. The formulation of claim 24, wherein in the Avelumab glycosylation said FA2 has a share of about 49% and said FA2G1 has a share of about 30%-about 35% of all glycan species.

26. The formulation of any one of claims 22-25, wherein the Avelumab glycosylation further comprises as minor glycan species A2 with a share of <5%, A2G1 with a share of <5%, A2G2 with a share of <5% and FA2G2 with a share of <7% of all glycan species.

27. The formulation of claim 26, wherein in the Avelumab glycosylation said A2 has a share of 3%-5%, said A2G1 has a share of <4%, said A2G2 has a share of <3% and said FA2G2 has a share of 5%-6% of all glycan species.

28. The formulation of claim 27, wherein in the Avelumab glycosylation said A2 has a share of about 3.5%-about 4.5%, said A2G1 has a share of about 0.5%-about 3.5%, said A2G2 has a share of <2.5% and said FA2G2 has a share of about 5.5% of all glycan species.

29. The formulation of any one of claims 22-28, wherein said Avelumab has the heavy chain sequence of (SEQ ID NO:2).

30. The formulation of any one of claims 1-29 which is for intravenous (IV) administration.

31. A vial containing the formulation of claim 30.

32. The vial of claim 31 which contains 200 mg avelumab in 10 mL of solution for a concentration of 20 mg/mL.

33. The vial of claim 31 or 32 which is a glass vial.

34. A method of treating cancer comprising administering the formulation of any one of claims 1-30 to a patient.

35. The method of claim 34 wherein the cancer is selected from non-small cell lung cancer, urothelial carcinoma, bladder cancer, mesothelioma, Merkel cell carcinoma, gastric or gastroesophageal junction cancer, ovarian cancer, breast cancer, thymoma, adenocarcinoma of the stomach, adrenocortical carcinoma, head and neck squamous cell carcinoma, renal cell carcinoma, melanoma, and/or classical Hodgkin's lymphoma.


SEQUENCE LISTING (3 sequences)

SEQ ID NO: 1 - protein, 450 aa, Artificial Sequence
  Heavy chain sequence of Avelumab
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 449 aa, Artificial Sequence
  Heavy chain sequence of Avelumab, lacking the C-terminal K
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 216 aa, Artificial Sequence
  Light chain sequence of Avelumab
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS